(19) United States
(12) Patent Application Publication
(10) Pub. No.: US 2021/0236575 A1
(43) Pub. Date: Aug. 5, 2021

(54) THERAPEUTIC COMBINATIONS OF CANNABINOIDS WITH CURCUMIN

(71) Applicant: EMERALD HEALTH THERAPEUTICS CANADA INC., Vancouver (CA)

(72) Inventors: Freydoun GARABAGI, Vancouver (CA); Nancy E. HARRISON, Vancouver (CA); Salam A. KADHIM, Vancouver (CA); Christopher WAGNER, Vancouver (CA); Andrew MCBRIDE, Vancouver (CA); Hardip SAHOTA, Vancouver (CA)

(21) Appl. No.: 17/271,147

(22) PCT Filed: Aug. 26, 2019
(86) PCT No.: PCT/IB2019/000953

§ 371 Date: Feb. 24, 2021

Related U.S. Application Data
(60) Provisional application No. 62/723,240, filed on Aug. 27, 2018.

(51) Int. Cl. A61K 36/185; A61K 31/05; A61K 31/352; A61K 31/12; A61K 9/00; A61K 9/48

(52) U.S. Cl. CPC A61K 36/185; A61K 31/05; A61K 31/352; A61K 2236/15; A61K 9/006; A61K 9/4891; A61K 9/0056; A61K 31/12

(57) ABSTRACT

Described herein are cannabinoid formulations in combination with curcumin for oral administration. Further described herein are methods for orally administering one or more cannabinoids to a subject in need thereof and manufacturing oral formulations as described herein.

### CROSS-REFERENCE TO RELATED APPLICATIONS

This application claims the benefit of U.S. Provisional Application No. 62/723,240, filed Aug. 27, 2018, which is incorporated herein by reference in its entirety.

### BACKGROUND OF THE INVENTION

Natural health products (NHPs), also known as dietary supplements, are manufactured products intended to supplement the diet when taken by mouth as a pill, capsule, tablet, or liquid. NHPs provide nutrients either extracted from food sources, animals or synthetically made, in order to restore or maintain good health and to increase the quantity of their consumption in the normal diet or because the nutrient is not available in a regular diet. NHPs are widely available. It is estimated that over 50% of North American citizens regularly consume one or more NHPs such as vitamins, amino acids, plants or plant extracts. In the United States and Canada, NHPs and dietary supplements are considered a subset of foods and are regulated accordingly. The European Commission has also established harmonized rules to ensure that NHPs are safe and properly labeled.

Cannabinoids from the plant genus Cannabis could be considered a type of natural health product, but historically they have not been legally available. The laws which have criminalized possession or use of Cannabis have been the primary restraint. These laws were put in place apparently to control the use of one specific cannabinoid, delta-9 tetrahydrocannabinol (THC), which causes a mild temporary psychotropic effect in users. But it is well known that dozens of other cannabinoids are also present in Cannabis, none of which have psychotropic effects, and which have, or potentially may have, beneficial pharmacological effects in humans. These alternate cannabinoids which are devoid of psychotropic effect include but are not limited to tetrahydrocannabinolic acid (THCA), cannabinolic acid (CBNA), cannabidiolic acid (CBDA) and cannabigerolic acid (CBGA) and the de-carboxylated derivatives cannabinol (CBN), cannabichromene (CBC), cannabidiol (CBD) and cannabigerol (CBG). With the de-criminalization of Cannabis in some jurisdictions, the opportunity for use of cannabinoids in diverse health regimens is becoming possible.

Cannabinoids bind to receptors in the body known as cannabinoid receptors which have been implicated in a variety of physiological functions, including appetite, pain, emotional behavior (mood), memory, and inflammation. There are currently two known well defined subtypes of cannabinoid receptors. The CB₁ receptor (CB1R) is expressed mainly in the brain (central nervous system or “CNS”), and also in the lungs, liver and kidneys. The CB₂ receptor (CB2R) is expressed mainly in the immune system and in hematopoietic or blood cells. There is mounting evidence that there are other novel cannabinoid receptors which are similar to CB1R and CB2R but which are unique.

The potential to use cannabinoids and NHPs in combination has yet to be fully explored. The invention herein is directed to novel combinations comprising A) one or more NHPs, and B) one or more cannabinoids, in oral formulation. Such products are useful as natural health products, dietary supplements, and for treatment of human diseases, conditions and disorders.

### SUMMARY OF THE INVENTION

Provided herein are cannabinoid and curcumin formulations, in combination, for oral administration.

In one aspect, described herein is an oral formulation comprising one or more cannabinoids selected from among the group consisting of: 0.1-750 mg tetrahydrocannabinolic acid (THCA), 0.1-100 mg tetrahydrocannabinol (THC), 0.1-750 mg cannabidiolic acid (CBDA), 0.1-750 mg cannabidiol (CBD), 0.1-750 mg cannabichromene (CBC), and 0.1-750 mg cannabigerol (CBG), 0.1-750 mg cannabichromene (CBC); and curcumin and/or another curcuminoid. In some embodiments, oral formulations are in a unit dosage form selected from the group consisting of a pill, tablet, capsule, film, wafer, lollipop, lozenge, oil, tincture, and syrup. In some embodiments, the formulation is an orally disintegrating pill, tablet, capsule, film, or wafer. In some embodiments, the formulation is a pill or tablet and further comprises an enteric coating for containing the one or more cannabinoids and the lipid carrier. In some embodiments, the formulation is a pill, tablet, or capsule, and further comprises an outer shell that is substantially opaque to one or both of ultraviolet and visible light. In some embodiments, the formulation further comprises a surfactant and/or a carrier oil, which may be an oil comprising an omega-3 to omega-6 ratio of about 1.0 or higher, about 1.5 or higher, about 2.0 or higher, or about 2.2. or higher. In some embodiments, the formulation further comprises a surfactant. In some embodiments, one or more of the cannabinoids is present in the form of an organic solvent-based extract of Cannabis. In some embodiments, at least one further cannabinoid selected from the group consisting of CBGA, CBC, and tetrahydrocannabivarin (THCV). In some embodiments, the formulation comprises CBD in an amount between 10-50 mg. In some embodiments, the formulation comprises 25 mg CBD. In some embodiments, the formulation comprises 500 mg CBD. In some embodiments, curcumin is present in an amount between about 200 mg and about 400 mg. In some embodiments, the dose of curcumin is present in an amount of about 200 mg, or about 400 mg. In some embodiments, the curcumin is present in the form of an organic solvent-based extract. In some embodiments, the cannabinoid is physically separated from the curcumin. In some embodiments, the cannabinoid is evenly dispersed within at least a portion of the oral formulation. In some embodiments, a signifier which signifies the cannabinoid dosage is associated directly with the oral formulation by embossing, or by colour, pattern or shape feature. In some embodiments, the signifier is adapted to be directly interpreted by a consumer and/or is a machine-readable code. In some embodiments, the oral formulation is contained in an individual blister pack sealed in an inert gas atmosphere comprising little or no oxygen.

In one aspect, described herein is a method of changing the level of a biomarker selected from IL-6, TNF-α, and C-reactive protein in the serum of an individual, the method comprising administering to the individual an oral formulation as described herein. In some embodiments, the individual is suffering from one or more diseases, conditions, or disorders selected from the group consisting of pain, inflammation, anxiety, depression, sleep disorders, insomnia, lack of energy, lack of alertness, weight gain, obesity, diabetes, Metabolic Syndrome, acute and anticipatory nausea, suppressed appetite, epilepsy, spasticity, schizophrenia, bi-polar disorder, cancer and neoplasia, chronic pain, osteoarthritic pain, bacterial and/or fungal infection and fibromyalgia. In some embodiments, the administering results in amelioration and/or treatment of one or more symptoms selected from the group consisting of pain, inflammation, anxiety, depression, sleep disorders, insomnia, lack of energy, lack of alertness, weight gain, obesity, diabetes, Metabolic Syndrome, acute and anticipatory nausea, suppressed appetite, epilepsy, spasticity, schizophrenia, bi-polar disorder, cancer and neoplasia, chronic pain, osteoarthritic pain, bacterial and/or fungal infection and fibromyalgia.

In one aspect, described herein is a method of treating an individual suffering from one or more diseases, conditions or disorders selected from the group consisting of pain, inflammation, anxiety, depression, sleep disorders, insomnia, lack of energy, lack of alertness, weight gain, obesity, diabetes, Metabolic Syndrome, acute and anticipatory nausea, suppressed appetite, epilepsy, spasticity, schizophrenia, bi-polar disorder, cancer and neoplasia, chronic pain, osteoarthritic pain, and fibromyalgia, the method comprising administration to the individual of a therapeutically effective amount of an oral formulation as described herein. In some embodiments, the disease or condition is associated with IL-6, TNF-α, or C-reactive protein. In some embodiments, the treating results in a change in a level of IL-6, TNF-α, or C-reactive protein in the serum of an individual. In some embodiments, the change is a reduction or an increase.

In one aspect, described herein is a method of manufacturing an oral formulation as described herein, comprising providing an organic extract of cannabinoids from cultivated Cannabis, measuring the concentration of one or more cannabinoids selected from the group consisting of THCA, THC, CBDA, CBC, and CBD in the organic extract, adjusting the concentration of one or more cannabinoids in the extract to prepare an adjusted extract within the concentration tolerance limits of a manufacturing specification for the oral formulation; and manufacturing the oral formulation with the adjusted extract.

In some embodiments, the formulation comprises: a unit dose or combination dose of cannabinoid(s) selected from the list consisting of (each cannabinoid milligram amount about or equal to):
THC (10 mg), CBD (10 mg), and curcumin (200 mg),
THC (10 mg), CBG (3 mg), and curcumin (200 mg),
THC (1 mg), CBD (25 mg), and curcumin (400 mg),
THC (10 mg), CBD (10 mg), and curcumin (323.4 mg),
THC (5 mg), CBD (20 mg), and curcumin (323.4 mg),
THC (10 mg), CBG (3 mg), and curcumin (323.4 mg),
THC (1 mg), CBD (10 mg), and curcumin (200 mg),
THC (1 mg), THCA (9 mg), and curcumin (200 mg),
THC (10 mg) and curcumin (200 mg),
THC (10 mg), CBD (10 mg), and curcumin (200 mg),
THC (10 mg), CBD (10 mg), and curcumin (400 mg),
THC (10 mg), CBD (10 mg), and curcumin (323.4 mg),
THC (1 mg), CBD (25 mg), and curcumin (323.4 mg),
THC (10 mg), CBG (3 mg), and curcumin (323.4 mg),
THC (10 mg), THCV (10 mg), and curcumin (200 mg),
THCV (10 mg), CBD (10 mg), and curcumin (200 mg),
THC (5 mg), CBD (10 mg), and curcumin (323.4 mg),
THC (5 mg), CBD (10 mg), and curcumin (200 mg),
THC (10 mg), CBG (3 mg), CBC (3 mg) and curcumin (200 mg),
THC (10 mg), CBG (3 mg), CBC (3 mg) and curcumin (400 mg),
THC (10 mg), CBG (2 mg), CBC (1 mg) and curcumin (180 mg),
.THC (6 mg), CBG (3 mg), CBC (3 mg) and curcumin (180 mg), and
CBD (6 mg), CBG (3 mg), CBC (3 mg) and curcumin (180 mg).

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts interactions between curcumin and human metabolic pathways.

FIG. 2 is a Venn diagram depicting the observation of the inventors that a set of 192 gene transcripts are independently activated by both curcumin and THC. Some of these gene transcripts are described in the text.

FIG. 3 is a Venn diagram depicting the observation of the inventors that a set of 69 gene transcripts are independently activated by both curcumin and CBD. Some of these gene transcripts are described in the text.

FIG. 4: A. is a dose-response curve representation (log [Curcumin concentration] (M) vs activity (Fold change)). Results are expressed as mean±S.E.M. (n=3). The IC50 value of Curcumin-THC combination was determined by fitting a dose response curve with nonlinear regression log [antagonist] (M) vs response (Fold change) using Prism 8 (GraphPad Software). B. Bar representation of CB1R activity (Fold change) of THC alone and curcumin-THC combination treatments. Results are expressed as mean±S.E.M. (n=3). Statistical significance was determined by non-parametric Kruskal-Wallis test—Uncorrected Dunn's multiple comparisons test. *, p<0.05. **, p<0.01.

FIG. 5: A. is a bar representation of CB1R activity (Fold change) of curcumin alone vs curcumin-THCA combination treatments. B. Bar representation of CB1R activity (Fold change) of curcumin alone vs curcumin-THC combination treatments. Results are expressed as mean±S.E.M. (n=3). Statistical significance was determined by non-parametric Kruskal-Wallis test—Uncorrected Dunn's multiple comparisons test. *, p<0.05. **, p<0.01.

FIG. 6: A. is a dose-response curve representation (log [Curcumin concentration] (M) vs activity (%)). Results are expressed as mean±S.E.M. (n=3). The IC50 values were determined by fitting a dose response curve with nonlinear regression log [antagonist] (M) vs normalized response (%) using Prism 8 (GraphPad Software). B. Bar representation of CB1R antagonist activity IC50 values. Results are expressed as mean±S.E.M. (n=3). Statistical significance was determined by non-parametric Kruskal-Wallis test—Uncorrected Dunn's multiple comparisons test. *, p<0.05. **, p<0.01.

FIG. 7: A. is a bar representation of CB1R activity (%) of curcumin alone vs curcumin-THC combination treatments. B. Bar representation of CB1R activity (%) of curcumin alone vs curcumin-THCA combination treatments. C. Bar representation of CB1R activity (%) of curcumin alone vs curcumin-CBD combination treatments. Results are expressed as mean±S.E.M. (n=3). Statistical significance was determined by non-parametric Kruskal-Wallis test—Uncorrected Dunn's multiple comparisons test. *, p<0.05. **, p<0.01.

FIG. 8: A. is a dose-response curve representation (log [Curcumin concentration] (M) vs activity (%)). Results are expressed as mean±S.E.M. (n=3). The EC50 values were determined by fitting a dose response curve with nonlinear regression log [antagonist] (M) vs normalized response (%) using Prism 8 (GraphPad Software). B. Bar representation of CB2 activity EC50 values. Results are expressed as mean±S.E.M. (n=3). Statistical significance was determined by non-parametric Kruskal-Wallis test—Uncorrected Dunn's multiple comparisons test. *, p<0.05. **, p<0.01.

FIG. 9: is a bar representation of CB1R activity (%) of curcumin alone vs curcumin-THC combination treatments. B. Bar representation of CB1R activity (%) of curcumin alone vs curcumin-THCA combination treatments. C. Bar representation of CB1R activity (%) of curcumin alone vs curcumin-CBD combination treatments. Results are expressed as mean±S.E.M. (n=3). Statistical significance was determined by non-parametric Kruskal-Wallis test—Uncorrected Dunn's multiple comparisons test. *, p<0.05. **, p<0.01.

FIG. 10: A. is a dose-response curve representation (log [Curcumin concentration] (M) vs NF-κB activity (%)). Results are expressed as mean±S.E.M. (n=3). Data were plotted by fitting a dose response curve with nonlinear regression log [antagonist] (M) vs normalized response (%) using Prism 8 (GraphPad Software. B. Bar representation of NF-κB activity IC50 values. Results are expressed as mean±S.E.M. (n=3). Statistical significance was determined by non-parametric Kruskal-Wallis test—Uncorrected Dunn's multiple comparisons test. *, p<0.05. **, p<0.01.

FIG. 11: A. is a bar representation of anti-NF-κB activity (%) of curcumin alone vs curcumin-THC combination treatments. B. Bar representation of anti-NF-κB activity (%) of Curcumin alone vs Curcumin-THCA combination treatments. Results are expressed as mean±S.E.M. (n=3). Statistical significance was determined by non-parametric Kruskal-Wallis test—Uncorrected Dunn's multiple comparisons test. *, p<0.05. **, p<0.01.

### DETAILED DESCRIPTION OF THE INVENTION

The invention herein provides novel oral formulations comprising a combination of one or more cannabinoids with the natural health product curcumin. The invention has a variety of advantages, including a surprising and synergistic effect for the treatment of human diseases, conditions and disorders. In particular, the oral formulation combinations provided herein demonstrate surprising and unexpected synergy for the treatment of a disease, condition or disorder selected from among pain, inflammation, anxiety, depression, sleep disorders, insomnia, lack of energy, lack of alertness, weight gain, obesity, diabetes, Metabolic Syndrome, acute and anticipatory nausea, suppressed appetite, epilepsy, spasticity, schizophrenia, bi-polar disorder, cancer and neoplasia, chronic pain, osteoarthritic pain, bacterial and fungal infection, fibromyalgia, and other disease, conditions and disorders disclosed herein.

As described herein, the invention is based on synergies identified by bioinformatics (overlapping sets of expressed genes induced by the combinations demonstrating mutually re-enforcing effects on specific biochemical pathways), and by testing the combinations in biological settings, including in vitro cell-based assays, in vivo pharmacokinetic/pharmacodynamic (PK/PD), biomarker assays and in animal models of complex disease. Product embodiments exemplifying the invention are also provided. The invention has an objective of increasing safety, confidence, and enhanced treatment of the noted diseases and disorders with the claimed oral combinations and particular unit dosage forms.

The claimed curcumin and cannabinoid compositions and their surprising synergy of effect has not been recognized by previous work, notably the disclosures published as WIPO publications WO2015171445 to Singh, WO2017066474 to Reynolds, WO2017168426 to Barzilay, and patents U.S. Pat. No. 9,744,132 to Bannister and U.S. Pat. No. 9,241,911 to Miller.

### Definitions

As used herein:

“Biomarker” means a measurable substance in the serum or tissue of an organism whose presence or level is indicative of a disease or condition. Biomarker presence or level will change (either increase or decrease) depending on the specific biomarker, and on the progress of the disease and the patient response to therapy.

“Cannabinoid” means any phytocannabinoid compound which 1) specifically binds to the human CB₁ receptor and/or CB₂ receptor under physiological conditions and which is 2) naturally synthesized by a plant (e.g. typically of the Cannabis species) or is a decarboxylated derivative thereof or is a liver metabolite thereof. Cannabinoids produced by Cannabis during its cultivation and growth include tetrahydrocannabinolic acid (THCA), cannabinolic acid (CBNA), cannabidiolic acid (CBDA) and cannabigerolic acid (CBGA). As used herein, cannabinoid also includes the corresponding decarboxylated moieties, tetrahydrocannabinol (THC), cannabinol (CBN), cannabidiol (CBD) and cannabigerol (CBG), (each of which may be derived from its parent compound by mild heating typically above 105° C.), and the corresponding liver metabolites that result upon oral consumption by humans of these compounds, such as but not limited to 11-OH-THC. Cannabinoids also include cannabichromene (CBC) and tetrahydrocannabivarin (THCV). Cannabinoids may be synthesized by chemical or biological methods. Phytocannabinoids may be distinguished from endocannabinoids which are chemically distinct, are synthesized in mammalian cells from lipids and other macromolecule precursors which are not phytocannabinoids, and are endogenous ligands of the CB₁ and/or CB₂ receptors.

“Cannabis” as used herein includes all members of the plant genus Cannabis, including without limitation C. sativa, C. indica, C. ruderalis, and hybrids thereof.

“Defined dose” means the dose of one or more active ingredients (typically cannabinoids) that has been selected during the production process and is signified to a consumer by a signifier associated with the oral formulation or Unit Dosage Form (UDF) of the invention.

“Natural Health Product” or “NHP” means a product which can be manufactured using sources from plants, algae, fungi or lichens, or other living matter. In some cases, an NHP may be dried plant matter, an extract, or a modification or a chemical derivative of a product found in the naturally occurring organism. NHPs are also known as dietary supplements or nutritional supplements in some contexts. They are typically regulated as foods and may be distinguished from drugs or pharmaceuticals which due to their powerful physiological effects and potential toxicities are more stringently regulated.

“Opaque” means tending to block transmission of visible light and/or UV-light, unless the context specifically indicates otherwise. “Substantially opaque” means substantially blocking including greater than or equal to than about 25%, 30%, 35%, 40%, 45%, 50%, 55%, 60%, 65%, 70%, 75%, 80%, 90%, 95%, 96%, 97%, 98%, 99%, and 100% blocking.

“Oral formulation” means a formulation which is conveniently administered orally to a human subject.

“Pharmacodynamic” parameters (PD) means dose-response relationships, that is, the relationships between a substances' plasma concentration and its effect.

“Pharmacokinetic” (PK) parameters are usually used to describe the rate of absorption of a substance into a biological system. Graphing a substance's serum concentration versus time reveals of the drug's basic PK properties: the maximum concentration the drug attains (Cₘₐₓ), the time at which this maximum concentration occurs (tₘₐₓ), and the area under the concentration-versus-time curve (AUC) which estimates total systemic exposure.

“Therapeutically effective amount” means an amount sufficient to elicit an objective or subjective therapeutic response to a disease or a condition in an individual. In the case of a unit dosage form, a therapeutically effective amount means one or more doses of the specific unit dosage form. For chronic conditions it may mean one or more doses per day or per week. In some embodiments a therapeutically effective amount will mean consumption of multiple UDF doses per day.

“Unit dosage form” or “UDF” means a physically fixed unit dose of a formulation which is conveniently consumed by a consumer in unit form (e.g. requires no measuring or adjusting of dosage before consumption). A consumer may consume one or more UDFs at a time.

### Key Ingredients of the Unit Dosage Form

Curcumin is a bright yellow chemical produced by some plants. It is the principal curcuminoid of turmeric (Curcuma longa), a member of the ginger family, Zingiberaceae. It is sold as an herbal supplement, cosmetics ingredient, food flavoring, and food coloring.

Chemically, curcumin is a diarylheptanoid, belonging to the group of curcuminoids, which are natural phenols responsible for turmeric's yellow color. It is a tautomeric compound existing in enolic form in organic solvents, and as a keto form in water. This invention relates primarily to curcumin but can equally be applied to alternative curcuminoids. Curcuminoids are polyphenolic pigments compounds in found in turmeric, including but not necessarily limited to diferuloylmethane (curcumin), demethoxycurcumin, bisdemethoxycurcumin.

Suitable dosage ranges for curcumin include 50-400 mg, about 100 mg, about 200 mg, and about 400 mg.

The unit dosage form of the invention further comprises a defined dose of one or more cannabinoids selected from among the group consisting of
0.1-750 mg tetrahydrocannabinolic acid (THCA),
0.1-100 mg tetrahydrocannabinol (THC),
0.1-750 mg cannabidiolic acid (CBDA),
0.1-750 mg cannabidiol (CBD),
0.1-750 mg cannabichromene (CBC), and
0.1-750 mg cannabigerol (CBG).

The cannabinoid(s) may be prepared as an extract of a cultivated Cannabis plant crop (as described further below), by a biosynthetic process or they may be synthetically prepared in a chemical process (as for example in patent applications WO2014134281, WO2015068052, WO2016030828 and others in the name of Full Spectrum Laboratories Limited (Dublin IE)). When prepared as an extract, the composition may also comprise terpenes and other organic molecules co-extracted in the process.

As will be described further below, the unit dosage form may also comprise diverse additional features which may include an anti-oxidant, other pharmaceutically acceptable additives, a carrier oil, an outer shell that is substantially opaque to one or both of ultraviolet and visible light, an enteric-coating, and/or a signifier which signifies the cannabinoid dosage of the unit dosage form, such as a signifier generated by embossing, or by colour, pattern or shape feature, which signifier may be adapted to be directly interpreted by a consumer and/or is a machine-readable code.

The inventors have identified the advantages of the proposed novel combinations based on a variety of technical assessments which demonstrate surprising and synergistic effects of the oral combinations on metabolic pathways relevant to treatment of specific human diseases and disorders. These assessments, described below, include 1) Identifying sets of expressed genes that are mutually activated by the combination (thus permitting stronger effects to be achieved using lower doses, as further described below); and 2) Identifying mutually interacting effects on specific biochemical pathways through chemical-protein interactions, herein identified as agonistic, allosteric and/or antagonistic effects.

Utility of the Invention

Based on the technical assessments disclosed herein, the oral formulation of the invention is provided as a therapeutic and natural health product agent for the treatment or amelioration in humans and other animals of a range of diseases, conditions and disorders. These diseases, conditions and disorders are selected from among the group consisting of pain, inflammation, anxiety, depression, sleep disorders, insomnia, lack of energy, lack of alertness, weight gain, obesity, diabetes, Metabolic Syndrome, acute and anticipatory nausea, suppressed appetite, epilepsy, spasticity, schizophrenia, bi-polar disorder, cancer and neoplasia, chronic pain, osteoarthritic pain, fibromyalgia, all the foregoing in acute and/or chronic presentation, and can be further used to induce appetite suppression and/or act as an anti-proliferative agent (for the treatment of neoplasia or cancer).

Additionally, the oral formulation can be used to treat or ameliorate Inflammatory Bowel Disease (IBD), Crohn's Disease (CD), arthritis (including osteoarthritis and rheumatoid arthritis, and other forms), cardiovascular Inflammation, ischemic heart disease, neuroprotection, and for use in treating muscle aches, persistent arthritis related pain, nociceptive and neuropathic pain, such as post-herpetic neuralgia, trigeminal neuralgia, diabetic neuralgia, and postoperative or posttraumatic pain, as well as endogenous depression, ADHD and symptoms of Parkinson's disease, Huntington's disease, Multiple Sclerosis, drug and alcohol dependence, asthma, allergic hypersensitivity, uveitis, eosinophilia, peritonitis, gastritis, exanthem, periodontitis, thrombocytopenia, pain agnosia, toxic shock syndrome, treatment of infectious diseases (including malaria, influenza and human immunodeficiency virus), anemia, lung diseases, neurological diseases, liver diseases, metabolic diseases, autoimmune diseases, cardiovascular diseases, hypoglycemia, wound healing, anti-microbial activities, psoriasis, ulcerative proctitis, ulcerative colitis, alveolar osteitis (dry socket), proliferative vitreoretinopathy (PVR), loss of appetite, abdominal cramps, diarrhea control, allodynia, medication-rebound headache, b-amyloid-induced neuroinflammation, reperfusion injury, autoimmune encephalomyelitis, acute lung injury, Alzheimer's disease, CNS inflammation, major depressive disorder, treatment resistant depression, anxiety disorders, post-traumatic stress disorder (PTSD), treatment of nightmares, PTSD-associated insomnia, other PTSD symptoms, toxic encephalopathy, cerebrovascular disease, hypertension, hyperglycemia, coronary artery disease, cardiomyopathy including hypertrophic and dilated cardiomyopathy, spinal cord injury, dementia, collagen disease, vasculitis, leukopenia and fatty liver disease, peripheral neuropathies (such as diabetic neuropathy, chemotherapy-induced peripheral neuropathy, carpal tunnel syndrome, sciatic pain, low-back pain, failed back surgery syndrome, dental pains, neuropathic pain in stroke, chronic pelvic pain, post-herpetic neuralgia, and vaginal pains), endometriosis-associated pain, neurohypophyseal diabetes, amnestic disorder, hypoglycemia, neonatal jaundice, diabetes insipidus, chronic kidney disease, ovarian hyperstimulation syndrome, Kuhnt Junius degeneration, capillary hemangioma, brain edema, cystinuria, portal hypertension, Coats' disease, and to provide immunosuppression.

The overlapping biochemical pathways of the combination of the invention also indicate that treatment is indicated for any disease, disorder or condition identified by abnormal levels (either excess or deficiency) of serum biochemical markers such as IL-6, IL-8, MCP-1, COX-2, IκBα, IL-1α, IL-1β, MKP-1, TNFα and C-reactive protein. Similarly, response to therapy can be observed by the impact on these serum biomarkers after administration of an oral formulation of the invention to a human subject.

### Production of Unit Dosage Forms of the Invention

Curcumin (CAS Number: 458-37-7) (Molecular weight: 368.38 g/mol) may be commercially sourced in powdered or liquid form. Suitable sources of powdered curcumin for embodiments of this invention include, but are not limited to, Millipore Sigma (Curcumin, SKU: C1386), Microingredients™ (Organic Curcumin, UPC No. 711301494190), BulkSupplements.Com (Curcumin 95% Natural Turmeric Extract Powder), and HerbaDiet (Turmeric Curcumin Powder 95% Extract). Suitable sources of liquid curcumin extract include, but are not limited to, Solgar (Full Spectrum Curcumin; UPC Nos. 033984595972, 033984547070, or 033984547087), Earthen Supplements (Liposomal Tumeric), NovaSol (NovaSol® Curcumin), Healthy Drops (Liposomal Curcumin)

### Powders:

Millipore Sigma (C1386) https://www.sigmaaldrich.com/catalog/product/sigma/c1386?lang=en&region=CA
Bulk Supplements, Curcumin 95% Natural Turmeric Extract Powder (https://www.bulksupplements.com/curcumin-turmeric-extract.html)
Microingredients™, Organic Curcumin https://microingredients.com/products/curcumin-95-turmeric-extract-powder-organic
HerbaDiet, Turmeric Curcumin 95% Extract Pure High Quality Powder https://herbadiet.in/products/turmeric-curcumin-powder-95-extract-pure-high-quality-powder-no-fillers-curcuminoids?variant=5901449134118
Xian Sciyu Biotech Co. Ltd, High natural turmeric root extract powder 95% curcumin hplc http://www.sybiotech.com/product/html/?51.html (https://www.alibaba.com/product-detail/High-natural-turmeric-root-extract-powder 60222265835.html?spm=a2700.7724838.2017115.25.701968aa5DRfjB&s=p)

### Liquids:

Solgar, Full Spectrum Curcumin https://www.solgar.com/SolgarProducts/Full-Spectrum-Curcumin.htm
NovaSol, NovaSOL® Curcumin http://novasolcurcumin.com/
Earthen Supplements, 100% Organic Pure Turmeric Curcumin Liquid Drops (liposomal turmeric) http://www.earthensupplements.com/products
Healthy drops, liposomal curcumin, non-GMO vegan https://www.healthydrops.net/product/liposomal-curcumin

The cannabinoids in the oral formulation of the present invention may be provided as dried plant matter, as extracts of plant matter, or as generated by chemical or biosynthetic synthesis. A valuable parameter for usefulness is on whether the format is flowable. The cannabinoid format is preferably efficient for mixing for loading into capsules, forming tablets, and the like. Stickiness of powders or high-viscosity of liquids can be a deterrent to efficient preparation. The invention therefore takes advantage cannabinoid formats that are sufficiently flowable for use in manufacturing the formulations described herein. Flowability of dried plant material may be enhanced by appropriate grinding and by addition of excipients, including but not limited to those described herein. Flowability of oil extracts may be enhanced by diluents, gliders and the like. Oil extracts with hydrophobic components may be mixed with powders to provide a dry flowable powder which can easily be mixed with other formulation components. (e.g., US Pat App. Publications 20170232210 and 20160243177, incorporated herein by reference). Alternatively, oil extracts may be spray dried with flowable particles to create a flowable powder format. In one preferred embodiment, extracts may be used to produce crystallized pure cannabinoids. Crystallized CBD can be prepared by high-vacuum treatment of extracts, as exemplified at https://www.leafscience.com/2017/11/06/cbd-isolate-powder/ (viewed 22 Aug. 2018). Crystallized forms can be rendered into a suitable flowable powder by techniques common in the capsule/tablet industry.

Curcumin in combination with cannabinoids is administered at dosages of 100, 200 and 400 mg (QD)×1 daily or (BID)×2 daily or (TID)×3 daily therapeutic regimens. The maximum curcumin dosage of 400 mg given twice a day has been shown to be beneficial for the relief of neuropathic pain associated with various pathological conditions. This dose corresponds to the recommended Health Canada requirements outlined in the Natural Health Products Ingredient Database (NHPID).

In some embodiments, the dosage of curcumin provided in the dosage forms of the present disclosure is about 200-400 mg, about 200 mg, or about 400 mg.

The oral combinations of the invention further comprise one or more cannabinoids selected from among the group consisting of:
0.1-750 mg tetrahydrocannabinolic acid (THCA),
0.1-100 mg tetrahydrocannabinol (THC),
0.1-750 mg cannabidiolic acid (CBDA),
0.1-750 mg cannabidiol (CBD),
0.1-750 mg cannabichromene (CBC), and
0.1-750 mg cannabigerol (CBG),

In some embodiments, the oral combinations may comprise a defined dose selected from the following ranges (which may be referred to as “low dose”): about 0 mg, 1 mg, 2, 3, 4, 5, 6, 7, 8, 9, or 10 mg, 20 mg, 30 mg, 40 mg, 50 mg, 60 mg, 70 mg, 80 mg, 90 mg, 100 mg, or any about any 1 mg interval between 0 mg and 100 mg THC, about 0 mg, 1 mg, 2, 3, 4, 5, 6, 7, 8, 9, or 10 mg, 20 mg, 30 mg, 40 mg, 50 mg, 60 mg, 70 mg, 80 mg, 90 mg, 100 mg, or any about any 1 mg interval between 0 mg and 100 mg THCA, about 0 mg, about 7 mg, about 75 mg, about 1 mg, 2, 3, 4, 5, 6, 7, 8, 9, or 10 mg, 20 mg, 30 mg, 40 mg, 50 mg, 60 mg, 70 mg, 80 mg, 90 mg, 100 mg, or about any 1 mg interval between 0 mg and 100 mg CBD, and/or about 0 mg, about 7 mg, about 75 mg, about 1 mg, 2, 3, 4, 5, 6, 7, 8, 9, or 10 mg, 20 mg, 30 mg, 40 mg, 50 mg, 60 mg, 70 mg, 80 mg, 90 mg, 100 mg, or about any 1 mg interval between 0 mg and 100 mg CBDA, or 10 mg, 20 mg, 30 mg, 40 mg, 50 mg, 60 mg, 70 mg, 80 mg, 90 mg, 100 mg, or about any 1 mg interval between 0 mg and 100 mg CBG, and/or about 0 mg, about 7 mg, about 75 mg, about 1 mg, 2, 3, 4, 5, 6, 7, 8, 9, or 10 mg, 20 mg, 30 mg, 40 mg, 50 mg, 60 mg, 70 mg, 80 mg, 90 mg, 100 mg, or about any 1 mg interval between 0 mg and 100 mg CBC. In some embodiments, the oral combinations comprise about 0 mg, 1 mg, 2, 3, 4, 5, 6, 7, 8, 9, or 10 mg, 20 mg, 30 mg, 40 mg, 50 mg, 60 mg, 70 mg, 80 mg, 90 mg, 100 mg, or about any 1 mg interval between 0 mg and 100 mg of one of the foregoing compounds. In some embodiments, the oral combinations of the present invention have defined dosages for more than one of the foregoing compounds. For example, in some embodiments, the oral combinations comprise from about 0 mg-1 mg, or any 0.1 mg interval therebetween THC, about 0 mg, about 9 mg, about 90 mg, about 1 mg, 2, 3, 4, 5, 6, 7, 8, 9, or 10 mg, 20 mg, 30 mg, 40 mg, 50 mg, 60 mg, 70 mg, 80 mg, 90 mg, 100 mg, or any about any 1 mg interval between 0 mg and 100 mg, about 0 mg, about 7 mg, about 75 mg, about 1 mg, 2, 3, 4, 5, 6, 7, 8, 9, or 10 mg, 20 mg, 30 mg, 40 mg, 50 mg, 60 mg, 70 mg, 80 mg, 90 mg, 100 mg, or any about any 1 mg interval between 0 mg and 100 mg CBD, and/or about 0 mg, about 7 mg, about 75 mg, about 1 mg, 2, 3, 4, 5, 6, 7, 8, 9, or 10 mg, 20 mg, 30 mg, 40 mg, 50 mg, 60 mg, 70 mg, 80 mg, 90 mg, 100 mg, or any about any 1 mg interval between 0 mg and 100 mg CBDA. In some embodiments, the oral combinations comprise from about 0 mg, about 9 mg, about 90 mg, about 1 mg, 2, 3, 4, 5, 6, 7, 8, 9, or 10 mg, 20 mg, 30 mg, 40 mg, 50 mg, 60 mg, 70 mg, 80 mg, 90 mg, 100 mg, or any about any 1 mg interval between 0 mg and 100 mg THC, 0 mg-1 mg THCA, or any 0.1 mg interval therebetween, about 0 mg, about 7 mg, about 75 mg, about 1 mg, 2, 3, 4, 5, 6, 7, 8, 9, or 10 mg, 20 mg, 30 mg, 40 mg, 50 mg, 60 mg, 70 mg, 80 mg, 90 mg, 100 mg, or any about any 1 mg interval between 0 mg and 100 mg CBD, and about 0 mg, about 7 mg, about 75 mg, about 1 mg, 2, 3, 4, 5, 6, 7, 8, 9, or 10 mg, 20 mg, 30 mg, 40 mg, 50 mg, 60 mg, 70 mg, 80 mg, 90 mg, 100 mg, or any about any 1 mg interval between 0 mg and 100 mg CBDA. In some embodiments, the compositions are substantially free of THC-type cannabinoid compounds. For example, in some embodiments the oral combinations comprise from about 0 mg-1 mg, or any 0.1 mg interval therebetween THC, 0 mg-1 mg THCA, or any 0.1 mg interval therebetween, about 0 mg, about 7 mg, about 75 mg, about 1 mg, 2, 3, 4, 5, 6, 7, 8, 9, or 10 mg, 20 mg, 30 mg, 40 mg, 50 mg, 60 mg, 70 mg, 80 mg, 90 mg, 100 mg, or any about any 1 mg interval between 0 mg and 100 mg CBD, and about 0 mg, about 7 mg, about 75 mg, about 1 mg, 2, 3, 4, 5, 6, 7, 8, 9, or 10 mg, 20 mg, 30 mg, 40 mg, 50 mg, 60 mg, 70 mg, 80 mg, 90 mg, 100 mg, or any about any 1 mg interval between 0 mg and 100 mg CBDA.

In some embodiments, the oral combinations may comprise a defined dose selected from the following ranges (which may be referred to as “high dose”): about 10 mg, 15, 20, 25, 30, 40, 50, 60, 70, 80, 90, or 100 mg, 200 mg, 300 mg, 400 mg, 500 mg, 600 mg, 700 mg, 750 mg, or any about any 10 mg interval between 0 mg and 750 mg THCA, about 0 mg, 1 mg, 2, 3, 4, 5, 6, 7, 8, 9, or 10 mg, 20 mg, 30 mg, 40 mg, 50 mg, 60 mg, 70 mg, 80 mg, 90 mg, 100 mg, or any about any 1 mg interval between 0 mg and 100 mg THC, about 10 mg, 15, 20, 25, 30, 40, 50, 60, 70, 80, 90, or 100 mg, 200 mg, 300 mg, 400 mg, 500 mg, 600 mg, 700 mg, 750 mg, or any about any 10 mg interval between 0 mg and 750 mg CBD, and/or about 10 mg, 15, 20, 25, 30, 40, 50, 60, 70, 80, 90, or 100 mg, 200 mg, 300 mg, 400 mg, 500 mg, 600 mg, 700 mg, 750 mg, or any about any 10 mg interval between 0 mg and 750 mg CBDA, or about 10 mg, 15, 20, 25, 30, 40, 50, 60, 70, 80, 90, or 100 mg, 200 mg, 300 mg, 400 mg, 500 mg, 600 mg, 700 mg, 750 mg, or any about any 10 mg interval between 0 mg and 750 mg CBG, and/or about 10 mg, 15, 20, 25, 30, 40, 50, 60, 70, 80, 90, or 100 mg, 200 mg, 300 mg, 400 mg, 500 mg, 600 mg, 700 mg, 750 mg, or any about any 10 mg interval between 0 mg and 750 mg CBC. In some “high dose” embodiments, the oral combinations comprise about 10 mg, 15, 20, 25, 30, 40, 50, 60, 70, 80, 90, or 100 mg, 200 mg, 300 mg, 400 mg, 500 mg, 600 mg, 700 mg, 750 mg, or any about 10 mg interval between 0 mg and 750 mg of one of the foregoing compounds. In some “high dose” embodiments, the oral combinations of the present invention have defined dosages of more than one of the cannabinoids. In some high dose embodiments, the compositions are substantially free of THC-type cannabinoid compounds. For example, in some embodiments the oral combinations comprise from about 0 mg-1 mg, or any 0.1 mg interval therebetween THC, 0 mg-1 mg THCA, or any 0.1 mg interval therebetween, plus CBD in the amount of about 0 mg, about 25 mg, about 75 mg, about 10 mg, 20, 30, 40, 50, 60, 70, 80, 90, or 100 mg, 200 mg, 300 mg, 400 mg, 500 mg, 600 mg, 700 mg, 750 mg, or any about 10 mg interval between 0 mg and 750 mg, and/or CBDA and/or CBC in the amount of about 0 mg, about 25 mg, about 75 mg, about 10 mg, 20, 30, 40, 50, 60, 70, 80, 90, or 100 mg, 200 mg, 300 mg, 400 mg, 500 mg, 600 mg, 700 mg, 750 mg.

In some embodiments, the oral combinations described herein comprise an “effective” amount of one or more of the cannabinoid ingredients described herein. The term “effective amount” refers to an amount of the one or more cannabinoid ingredients sufficient to induce a response in an individual user, either subjectively or objectively determined. An effective amount also means an amount of the one or more cannabinoid ingredients that is needed to provide a desired level of cannabinoid(s) in the bloodstream of an individual user to provide an anticipated physiological response. An effective amount of a cannabinoid ingredient can be administered in one administration, or through multiple administrations of an amount that totals an effective amount, preferably within a 24-hour period. It is understood that the effective amount can be the result of empirical and/or individualized (case-by-case) determination on the part of the individual user. For example, a therapeutically effective amount of said one or more cannabinoid ingredients may be in the range of about 1 mg to 2,000 mg, or any 1 mg or 10 mg interval therebetween total cannabinoids per day.

In some low dose embodiments, an effective amount of said one or more cannabinoid ingredients may be in the range of about 1 mg-5 mg, or any 1 mg or 0.1 mg interval therebetween per day. For example, for an adult, about 1-2 mg, or 0.1 mg interval therebetween, per day total of THC may provide a very low end dose below the psychoactive threshold.

In some embodiments, an effective amount of THC may be in the range of about 5 mg-25 mg, or any 1 mg interval therebetween. For example, most vapers inhale about 10 to 30 mg of THC to establish a mild, temporary, psychoactive effect. In a high dose embodiment the oral formulation may contain THC in an amount of 25 mg to 100 mg.

In some embodiments, a composition of the present invention may comprise THCA in an amount between 5-200 mg, THC in an amount less than 1.0 mg, and CBDA in an amount between 0.1-600 mg, and have a total mass of 100-750 mg.

In some embodiments, a composition of the present invention may comprise THCA in an amount less than 5.0 mg, THC in an amount between 5-30 mg, and CBD in an amount between 0.1-600 mg, and have a total mass of 100-750 mg.

In some embodiments, a composition of the present invention may comprise THCA in an amount less than 1.0 mg, THC in an amount less than 1.0 mg, and CBD in an amount between 5-600 mg, and have a total mass of 100-750 mg.

In some embodiments, a composition of the present invention may comprise THCA in an amount less than 1.0 mg, THC in an amount less than 1.0 mg, and CBG in an amount between 5-600 mg, and have a total mass of 100-750 mg.

In some embodiments, an effective amount of CBD or CBC for treating conditions or disorders disclosed elsewhere herein may be in the low dose range of about 0 mg, about 7 mg, about 75 mg, about 1 mg, 2, 3, 4, 5, 6, 7, 8, 9, or 10 mg, 20 mg, 30 mg, 40 mg, 50 mg, 60 mg, 70 mg, 80 mg, 90 mg, 100 mg, or any about any 1 mg interval between 0 mg and 100 mg per day. Preferably, the low dose amount of CBD may be about 50 mg per day. For example, a recommended CBD or CBC dosage standard may be about 25 mg of CBD or CBC taken twice a day.

Alternatively, in some embodiments, an effective amount of CBD or CBC for treating conditions or disorders disclosed elsewhere herein may be in the high dose range of about 50-2000 mg/day or higher. Such effective amounts may be provided by ingestion of multiple oral dosage forms comprising CBD or CBC in the amount of, about 50 mg, about 75 mg, about 100 mg, 200 mg, 250 mg, 300 mg, 400 mg, 500 mg, 600 mg, 700 mg, 750 mg, or any about any 10 mg interval between 100 mg and 750 mg.

In some embodiments, an effective amount of THCA may be in the range of about 0 mg, about 9 mg, about 90 mg, about 1 mg, 2, 3, 4, 5, 6, 7, 8, 9, or 10 mg, 20 mg, 30 mg, 40 mg, 50 mg, 60 mg, 70 mg, 80 mg, 90 mg, 100 mg, or any about any 1 mg interval between 0 mg and 100 mg.

In some embodiments, only one cannabinoid will be present at a physiologically relevant level, in other embodiments two or more cannabinoids may be present at physiologically relevant levels. The second cannabinoid may be one previously cited or may be an alternative cannabinoid which may also be isolated from or extracted from Cannabis, or may be a simple chemical derivative thereof. Table 1 provides examples for preferred embodiments.

TABLE 1
Preferred cannabinoid doses (single or in combination) of the UDF.
Table 1A - 250 mg capsule (low dose)

| | Pre- | | | | | | | | |
|---|---|---|---|---|---|---|---|---|---|
| Form | dominant | Active Ingredient (mg/cap) | | | | | | | |
| # | Cannabinoid(s) | THCA | THC | CBDA | CBD | CBG | CBC | THCV | Indication |
| 1 | THCA | 25 | 2 | | | | | | Pain, Acute and anticipatory nausea; |
| | | | | | | | | | Obesity, Metabolic Syndrome |
| 2 | THC | | 25 | | | | | | Pain, Appetite enhancement |
| 3 | CBDA | 1 | | 25 | 2 | | | | Acute and anticipatory nausea |
| 4 | CBD | | 1 | | 25 | | | | Anxiety, Sleep |
| 5 | THCA:CBDA | 25 | 2 | 25 | 2 | | | | Acute and anticipatory nausea; |
| | | | | | | | | | Obesity, Metabolic Syndrome |
| 6 | THCA:CBD | 25 | 2 | | 25 | | | | Pain; Anxiety; Sleep; Obesity, |
| | | | | | | | | | Metabolic Syndrome |
| 7 | THC:CBD | | 25 | | 25 | | | | Pain; Anxiety; Sleep |
| 8 | THC:CBD | | 25 | | 2 | | | | Energy |
| 9 | CBD:CBG:CBC | | 1 | | 25 | 25 | 25 | | Osteoarthritic Pain |
| 10 | THC:THCV | | 25 | | | | | 25 | Energy |

Table 1B - 250 mg capsule (low dose; 10 mg THC maximum)

| | Pre- | | | | | | | | |
|---|---|---|---|---|---|---|---|---|---|
| Form | dominant | | | | | | | | |
| # | Cannabinoid(s) | THCA | THC | CBDA | CBD | CBG | CBC | THCV | Indication |
| 11 | THCA | 9 | 1 | | | | | | Pain, Acute and anticipatory nausea; |
| | | | | | | | | | Obesity, Metabolic Syndrome |
| 12 | THC | | 10 | | | | | | Pain, Appetite enhancement |
| 13 | THCA:CBDA | 9 | 1 | 9 | 1 | | | | Acute and anticipatory nausea; |
| | | | | | | | | | Obesity, Metabolic Syndrome |
| 14 | THCA:CBD | 9 | 1 | | 10 | | | | Pain; Anxiety; Sleep |
| 15 | THC:CBD | | 10 | | 10 | | | | Pain; Anxiety; Sleep |
| 16 | THC:CBD | | 10 | | 1 | | | | Energy |
| 17 | THC:THCV | | 10 | | | | | 10 | Energy |

Table 1C - 1000 mg capsule (high dose)

| | Pre- | | | | | | | | |
|---|---|---|---|---|---|---|---|---|---|
| Form | dominant | | | | | | | | |
| # | Cannabinoid(s) | THCA | THC | CBDA | CBD | CBG | CBC | THCV | Indication/function |
| 19 | THCA | 600 | | | | | | | Pain; Acute and anticipatory |
| | | | | | | | | | nausea; Obesity, Metabolic |
| | | | | | | | | | Syndrome |
| 20 | THCA:THC | 600 | 60 | | | | | | Pain; Obesity, Metabolic |
| | | | | | | | | | Syndrome |
| 21 | THC | | 100 | | | | | | Pain; |
| 22 | CBDA | | | 600 | | | | | Acute and anticipatory nausea |
| 23 | CBDA:CBD | 25 | | 600 | 60 | | | | Acute and anticipatory nausea |
| 24 | CBD | | | | 100 | | | | Anti-epileptic |
| 25 | CBD | | 4 | | 100 | | | | Anti-epileptic |
| 26 | CBD | | | | 600 | | | | Anti-epileptic |
| 27 | CBD | | 25 | | 600 | | | | Chronic Pain; Inflammation; |
| | | | | | | | | | Schizophrenia; Cancer Anti- |
| | | | | | | | | | proliferative |
| 28 | CBG | | | | | 600 | | | Cancer Anti-proliferative; |
| | | | | | | | | | Antimicrobial; bone stimulant |
| 29 | THCA:CBDA | 300 | | 300 | | | | | Acute and anticipatory nausea; |
| | | | | | | | | | Obesity, Metabolic Syndrome |
| 30 | THCA:CBDA | 300 | 30 | 300 | 30 | | | | Acute and anticipatory nausea, |
| | | | | | | | | | Obesity, Metabolic Syndrome |
| 31 | THCA:CBD | 300 | | | 300 | | | | Pain; |
| 32 | THCA:CBD | 300 | 30 | | 300 | | | | Pain; |
| 33 | THC:CBD | | 100 | | 100 | | | | Pain; Spasticity; |
| 34 | THC:CBD | | 100 | | 30 | | | | Pain; |
| 35 | THC:CBG | | 300 | | | 300 | | | Pain; Cancer Anti-proliferative |
| 36 | THC:CBC | | 300 | | | | 300 | | Pain; Anti-Inflammatory |
| 37 | CBD:CBG | | | | 300 | 300 | | | Pain; Cancer Anti-proliferative |
| 38 | CBD:CBC | | | | 300 | | 300 | | Pain; Anti-Inflammatory |
| 39 | CBD:CBG:CBC | | | | 300 | 300 | 300 | | Osteoarthritic Pain; Anti- |
| | | | | | | | | | proliferative |
| 40 | CBD:CBG:CBC | | 10 | | 250 | 250 | 250 | | Osteoarthritic Pain; Anti- |
| | | | | | | | | | proliferative |
| 41 | THC:THCV | | 100 | | | | | 500 | Pain; |
| 42 | CBD:THCV | | | | 300 | | | 300 | Appetite suppression; |
| 43 | CBD:THCV | | | | 100 | | | 100 | Anti-epileptic |

The precise amount of cannabinoid required for a therapeutically effective dose in an individual will depend upon numerous factors, e.g. type of cannabinoid(s) and type of natural health product, and the synergistic effect of the combination. This disclosure provides UDFs suitable to obtain a therapeutically effective dose which can be determined subjectively by the user or objectively by methods known to those skilled in the art.

An achievement of the invention is that by using the UDF of the invention, users and medical advisors for the first time have knowledge of and certainty with the exact doses of cannabinoid they are employing with curcumin. This is preferably achieved with a signifier identifying dosage of one or more components, as detailed further below.

### Source and Quality of Cannabinoid

The cannabinoid(s) may be prepared by a variety of methods. It may be provided in the original plant form, preferably dried and cured into a flowable powder suitable for encapsulating. An alternative preferred method is by extraction from a cultivated Cannabis crop. Organic extraction is a preferred method, although aqueous extraction, typically employed to prepare hashish, is also possible. Organic extraction can be performed with a wide variety of organic solvents or super-critical carbon dioxide, and at a variety of temperatures and under a variety of conditions. (Fairbairn and Liebmann (1973) J. Pharm. Pharmac. 25:150-155; Romano and Hazekamp (2013) Cannabinoids. 1(1)-1-11; Rovetto and Aieta (2017). J. Supercritical Fluids. 129: 16-27.), each of which references is incorporated herein by reference in its entirety. The resulting organic solvent-based extract can be, at room temperature, a liquid oil, or solid form wax, budder or shatter depending on the conditions employed (which significantly impact the other plant alkyloids and polymers extracted by the process). Historically, less than 50% of cannabinoids were extracted from dried plant material. (Fairbairn and Liebmann (1973)). Modern techniques may extract over 90%.

The unpredictability of cultivated Cannabis is another challenge that must be overcome. As is well known, the most common varieties C. sativa, C. indica and C. ruderalis, have distinct (but overlapping) ranges of cannabinoids. Varieties and strains which are crossed or hybridized generate further different cannabinoid ratios. And, the cannabinoid ratios and overall amounts within a single variety are strongly influenced by the conditions of cultivation, especially light cycle, temperature, soil condition, nutrient availability, timing of harvest and pathogen exposure. The result is that a cultivated Cannabis can have, by dry weight, anywhere from 0% up to greater than 30% of selected cannabinoids, and the ratios between individual cannabinoids can be highly diverse.

Preferred Cannabis sp. cultivars for use preparing cannabinoid extracts include Time Warp A3 (Hybrid, THC); Island Honey; Blue God; OGMB; Critical Call Mist; Sensi Little Twin; Nukem; Sensi Big Twin; Blueberry Kush; Afghani Kush; Crystal Kush; Big Bud XXL; Ocean Pearl; Critical Kush; K8; MK Tonic; Saltspring King; Purple X Chemo; Hash Plant (Indica, THC); White Rhino (Indica, THC); Master Kush (Indica, THC); Headband (Hybrid, THC); AK47 (Hybrid, THC); Armageddon (Hybrid, THC); Critical Kali Mist (Sativa, THC); Blue Cheese (Indica, THC); CBD Shark (Indica, THC); Sour Diesel (Sativa, THC); Durban Poison (Sativa, THC); Blue Cheese (Indica, THC); Acapulco Gold; Afghani; African; Cambodian red; Columbian; Hawaiian; Jamaican gold; Mexican red; Panama red; Thai stick; Amnesia; AK-47; Amnesia Haze; Blueberry; Blue Dream; Bubba Kush; Bubblegum; Critical Mass; Durban Poison; Gorilla Glue; Haze; Hindu Kush; Jack Herer; Maui Waui; Northern Lights; OG Kush; Purple Haze; and Skunk. Preferred for cultivation in Canada are: Altair, Angie, CS, Carmagnola, Carmen, Deni, ESTA-1, FINOLA, Fasamo, Fedrina 74, Felina 34, Fibranova, Fibriko Fibrimon 24, Fibrimon 56, Georgina, GranMa, Grandi, Judy, Katani, Kompolti, Kompolti Hibrid TC, Kompolti Sargaszaru, Laura Secord, Lovrin 110, Martha, Petera, Picolo, Quida, UC-RGM, Uniko B, Victoria, and Yvonne.

Preferred Cannabis sp. cultivars for use in preparing CBD extracts that contain little or no THC or THCA include: Charlotte's Web, Island Mist (Sativa, CBD), ACDC (Hybrid, CDB), Harle TSU (Hybrid, CBD), and cultivars approved in Canada including CFX-1, CFX-2, CRS-1, Canda, Crag, Joey, USO 14, USO 31, X-59 (Hemp Nut), Delores, Silesia, Alyssa, Zolotonosha 11, Anka, Jutta, CanMa, and Ferimon.

Another critical aspect of the cannabinoid preparation is that during the cultivation phase, Cannabis naturally synthesizes tetrahydrocannabinolic acid (THCA) and cannabidiolic acid (CBDA). These compounds convert respectively to THC (the primary psychoactive cannabinoid), and CBD (the non-psychoactive analgesic and anti-inflammatory cannabinoid) via decarboxylation. Decarboxylation may be induced by heating over 105° C. and/or by exposure to ultraviolet (UV) light. Significantly, gastric acids do not decarboxylate THCA or CBDA. (See Wang et al. (2016) Decarboxylation Study of Acidic Cannabinoids: A Novel Approach Using Ultra-High-Performance Supercritical Fluid Chromatography/Photodiode Array-Mass Spectrometry. Cannabis Cannabinoid Res.; 1(1): 262-271.) Therefore, a critical aspect of extracted cannabinoids is quality control on whether the harvested crop has been subjected to decarboxylating conditions that would decarboxylate THCA and CBDA to THC and CBD, respectively.

Additionally, minor cannabinoids may be present in certain strains at therapeutically useful levels. Cannabichromene (CBC) is a non-psychoactive cannabinoid widely considered to interact with the endocannabinoid system (ECS) through stimulation of the body's naturally occurring endocannabinoids, anandamide and 2-AG, and is a known agonist to TRPV1 and TRPA1 receptors (A. A. Izzo et al.: Br. J. Pharmacol. 166, 1444 (2012)). Additionally CBC is thought to be a selective CB2 receptor agonist which may have therapeutic implications for the treatment of pain and inflammatory conditions through CB2-mediated regulatory pathways (M. Udoh et al.: Br. J. Pharmacol. (2019). Furthermore, CBC co-administered with THC produced an enhanced anti-inflammatory effect, suggesting a potential pharmacokinetic interaction between the two molecules (G. T. DeLong et al.: Drug Alcohol Depend. 112, 126 (2010). CBC has been identified as a molecule of interest for various therapeutic applications including pain, inflammation, digestive and gastrointestinal disorders. Additionally, it is known to have antibacterial and antifungal effects, and could potentially contribute to the regeneration of brain cells, which possibly has implications in the treatments of multiple sclerosis, fibromyalgia, dementia, Alzheimer's and other neurodegenerative related conditions.

Because of the unpredictability of Cannabis cultivation, the invention requires that all extract preparations of cannabinoid(s) be analyzed to determine the precise concentrations of relevant cannabinoids, especially THCA, THC, CBDA, CBD, CBC and CBG for use in preparing unit dosage forms of the invention.

### Pharmaco-Analytical Testing of Cannabinoid(S) for Use in Preparation of the Defined Dose Oral Combination

Any chemical analytical method may be employed to determine the amount of the cannabinoids in the preparation used for formulating the UDF. Many methods are available to those skilled in the art, such as those found in Thomas, BF and El Sohly, M 2015 “The Analytical Chemistry of Cannabis: Quality Assessment, Assurance, and Regulation of Medicinal Marijuana and Cannabinoid Preparations” (Elsevier). See also Wang et al. (2016) Decarboxylation Study of Acidic Cannabinoids: A Novel Approach Using Ultra-High-Performance Supercritical Fluid Chromatography/Photodiode Array-Mass Spectrometry. Cannabis Cannabinoid Res.; 1(1): 262-271; and Wang et al. (2017) Quantitative Determination of Cannabinoids in Cannabis and Cannabis Products Using Ultra-High-Performance Supercritical Fluid Chromatography and Diode Array/Mass Spectrometric Detection. J Forensic Sci.; 62(3):602-611.) A particularly recommended approach is found at Mudge et al. (2017) Anal Bioanal Chem (2017) 409:3153-3163 DOI 10.1007/s00216-017-0256-3.

Testing may be performed to identify the cannabinoid content of the ground dried plant form, any other solid form or a liquid extract preparation.

Testing may be required at one step or at multiple steps in the production process. It may be first performed as a batch assay to ascertain amounts of relevant cannabinoids from a particular harvest or extraction process. The representative sample and measurement technique must be sufficient to represent all samples of the process batch within the degree of variability tolerated by the overall process, namely+/−25% of the defined dose of each cannabinoid. Depending on the result, the cannabinoid preparation may need to be adjusted (either diluted or concentrated) to generate a cannabinoid preparation to meet the tolerance range of volume/dose range for manufacturing specifications of the UDF. The operator will have available a variety of cannabinoid diluents or concentrating processes and/or oils of known cannabinoid concentrations to adjust the preparation. Often only one cannabinoid will need to be added, the others being already at satisfactory levels. The operator can determine by simple algorithm which amount of which additives and/or which concentration steps are required to obtain the desired preparation. The final preparation of cannabinoid may again be chemically analyzed. Any final preparation which is not within tolerance levels is discarded or re-processed until desired cannabinoid levels are obtained. The final tolerance level is within +/−25%, preferably within +/−20%, +/−15%, +/−10%, +/−5%, +/−2% and most preferably within +/−about 1% of the desired in-going amount of each defined dose cannabinoid in the preparation used for formulating the UDF. Alternatively stated, the UDF is expected to contain a dose of from 80% to 120% of the amount stated on product label. Preferably the range will be significantly more precise.

Where the method of the invention requires a Cannabis grinding step, this step must be executed properly to achieve the defined dose of the invention. Grinding risks degradation of the product by generation of heat, by clumping of sticky materials, and by loss of material to the grinding instrument. All aspects must be carefully controlled to achieve superior results.

Preferably, Cannabis will be ground to sieve through a mesh of not larger than about 0.1 mm to about 3 mm, or any 0.1 mm increment therebetween, more preferably not larger than about 1.5, mm in any surface dimension. In some embodiments, the sieve comprises 30, 60, or 120 mesh. In some embodiments, the sieve comprises an average opening size of about 0.595 mm, about 0.250 mm, or about 0.125 mm. Cannabis material may include, without limitation, the leaves, inflorescences, flowers, or buds of one or more Cannabis plants. The grinding step may use any grinding method or methods, such as hand grinding, machine grinding, or use of a chipper or mulcher, provided that a consistent milled size product as homogenous as possible is generated without degradation. Degradation can occur through generation of heat during the grinding process and should be carefully controlled.

### Biosynthetic Production of Cannabinoids

Biosynthesis of cannabinoids by engineered microbial strains (e.g. using eukaryotes, including but not limited to Yeast, Pichia, microalgae, or plant cell-based systems; or prokaryotes including but not limited to E. coli) is an alternative strategy for the production of cannabinoids. The identification of the enzymes involved in cannabinoid biosynthetic pathways enables the reconstruction of the pathway using a suitable heterologous host system. In addition, enzymes can be reconstituted in a cell-extract or a cell-free system to generate cannabinoids from precursor molecules. A synthetic biology approach can be especially interesting for the production of less-abundant cannabinoids. A wide variety of biosynthetic pathways for cannabinoids are set out in Carvahlo et al. (2017) FEMS Yeast Research, 17, 2017, fox037 doi: 10.1093/femsyr/fox037.

### Analytical Identification of Terpenes and Other Cannabis Plant Components in the Cannabinoid Preparation

Depending on the extraction process employed, a variety of other plant constituents may be extracted from Cannabis along with the cannabinoids. It may be desirable to identify and confirm concentrations of these components. Terpenes, chlorophylls, other alkaloids and macromolecules may also be detected by gas chromatography, mass spectroscopy, high-pressure liquid chromatography, or techniques standard in the art.

In certain embodiments, the signifier used with the unit dosage form product may also indicate the defined dose of such additional plant components.

Additional NHPs: Certain embodiments of the invention incorporate an additional natural health product or dietary supplement, or an alternate form of curcumin. A wide range of such products may be included. They may provide further surprising and synergistic advantages to the composition, or they may simply enhance the product subjectively or objectively. While any NHP or dietary supplement that is safe for human consumption at the dosage provided could be employed, most preferred for the invention are selected from among one or more of: turmeric, Palmitoylethanolamine (PEA), DL-Phenylalanine (DLPA), Boswellic Acid (AKBA), Gamma aminobutyric acid (GABA), Acetyl-L-carnitine (ALC), Alpha lipoic acid (ALA), 5-hydroxytryptophan (5-HTP), Echinicaea, Lavender, and Melatonin. Further alternatives include Ashwagandha (root), St. John's Wort Extract (aerial), Valerian (root), Rhodiola Rosea Extract (root), Lemon Balm Extract (leaf), L-Theanine, Passion Flower (herb), cyracos, gotu kola, chamomile, skullcap, roseroot, ginkgo, Iranian borage, milk thistle, bitter orange, sage, L-lysine, L-arginine, Hops, Green Tea, calcium-magnesium, Vitamin A (beta carotene), Magnolia officinalis, Vitamin D3, Pyridoxal-5-phosphate (P5P), St Johns wort, Cayenne, pepper, wasabi, evening primrose, Arnica Oil, Ephedra, White Willow, Ginger, Cinnamon, Peppermint Oil, Thiamin (Vitamin B1) (as thiamin mononitrate), Riboflavin (Vitamin B2), Niacin (Vitamin B3) (as nicotinamide), Vitamin B6 (pyridoxine HCl), Vitamin B12 (cyanocobalamin), California Poppy, Mullein Verbascum thapsus (L.), Kava Piper methysticum (G. Forst.), Linden Tilia cordata (Mill.), Catnip Nepeta cataria (L.), Magnesium, D-Ribose, Rhodiola Rosea, caffine, Branched-Chain Amino Acids Wheatgrass Shot, Cordyceps, Schisandra Berry, Siberian Ginseng (Eleuthero root), Yerba Mate Tea, Spirulina, Maca Root, Reishi Mushroom, Probiotics, Astragalus, He Shou Wu (Fallopia multiflora or Polygonum multiflorum), Cola acuminata (Kola nut), Vitamin C, Centella asiatica (Gotu kola), L-tryosine, Glycine, Pinine, Alpha-pinene, SAMe, DHEA, Co enzyme q10 and glutathione. The additional NHP may also be selected from among the Essential Oils: Anise (Pimpinella anisum(L.)), Basil (Ocimum basilicum(L.)), Bay (Laurus nobilis(L.)), Bergamot (Citrus aurantium var. bergamia (Risso)), Chamomile (German) (Matricaria recutita(L.)), Chamomile (Roman) (Chamaemelum nobile (L.) All.), Coriander (Coriandrum sativum (L.)), Lavender (Lavandula angustifolia (Mill.)), Neroli (Citrus aurantium (L.) var. amara), Rose (Rosa damascena (Mill.)), Sandalwood (Santalum album(L.)), Thyme (Thymus vulgaris (L.)), Vetiver (Vetiveria zizanioides(Nash),) Yarrow (Achillea millefolium(L.)), and Ylang ylang (Cananga odorata(Lam.) var. genuine).

The oral formulation of the invention may optionally further comprise additional components such as but not limited to carrier oils, surfactants, stabilizers, anti-oxidants, preservatives and excipients, as further described below.

A wide variety of carrier oils may be employed to dissolve, solubilize or otherwise formulate the components of the invention into a liquid or semi-solid formulation suitable for manufacturing the oral formulation and unit dosage forms of the invention. Carrier oils may comprise short chain, medium chain and/or long chain fatty acids. Typically, carrier oils comprise, by mass, from about 1% to about 99%, about 5% to about 93%, about 25% to about 85%, and optionally about 5% to about 35% of the UDF. The oils may be formulated with the cannabinoid and/or the NHP components of the invention through any known formulation process, including but not limited to oil-in-water emulsions, liposomes (e.g. fully encapsulated or aggregated), and nanoparticles. Omega-3, omega-6 and w-9 fatty acids are desirable. In some embodiments, the carrier oils comprise ratios of omega-3 oils to omega-6 oils (on a weight per weight basis) of 1.0 or higher, including ratios of 1.5, 2.0, 2.2, and 3.75. Omega-3 oils include essential oils such as EPA, DHA and alpha lipoic acid. The carrier oils are preferably extracts of plants or plant parts such as nuts, berries, roots, flowers of plants. All carrier oils employed will be safe for human consumption at the dosages provided. For use in a softgel or hardgel of the invention, oil-based preparations may be mixed with a surfactant, such as but not limited to Labrasol™. Surfactants, typically added at about 1-10% by weight, allow the formulation to convert to an emulsion upon exposure to the aqueous environment of the gut. Emulsions can be useful to enhance bioavailability of active ingredients.

The term “antioxidant” is used herein includes any compound or combination of compounds that prevent or slow down oxidation of components caused by the damaging reactive oxygen species (ROS). Any of the known antioxidants may be used, including but not limited to tocopherols, phospholipids (PL), phytosterols, phycocyanin, vitamins E, A and C, betacarotene, coenzyme Q10, fatty acids omega-3, omega-6 and w-9, phytoantioxidants such as polyphenols, terpenes as butylated hydroxyanisole (BHA), butylated hydroxytoluene (BHT), propyl gallate, lecithin, sesamin, sesamol, sesamolin, α-tocopherol, γ-tocopherol, salicylic acid, ascorbic acid, ascorbyl palmitate, fumaric acid, malic acid, sodium ascorbate and sodium meta-bisulphite, as well as chelating agents such as disodium EDTA. Pharmaceutically acceptable nutraceutical dietary supplements may also be employed as anti-oxidants including plants, alga, and lichen and may include one or more extracts of honeybee propolis, red clover, soybean, caper, almond, milk thistle, green tea, pomegranate, orange red, grape seed, bilberry, fo-ti root, ginseng, English ivy, red algae, brown algae, green algae and lichens.

Selection of excipients for the unit dosage form is a skill well known to those in the art of pharmaceutical dosage forms. Excipients may include one or more pharmaceutically acceptable carriers, diluents, fillers, hinders, lubricants, glidants, disintegrants, bulking agents, flavourants or any combination thereof. Non-limiting examples of suitable pharmaceutically acceptable carriers, diluents or fillers for use in the invention include lactose (for example, spray-dried lactose, .alpha.-lactose, .beta.-lactose), or other commercially available forms of lactose, lactitol, saccharose, sorbitol, mannitol, dextrates, dextrins, dextrose, maltodextrin, croscarmellose sodium, microcrystalline cellulose (for example, microcrystalline cellulose available under the trade mark Avicel), hydroxypropylcellulose, L-hydroxypropylcellulose (low substituted), hydroxypropyl methylcellulose (HPMC), methylcellulose polymers (such as, for example, Methocel A, Methocel A4C, Methocel A15C, Methocel A4M), hydroxyethylcellulose, sodium carboxymethylcellulose, carboxymethylene, carboxymethyl hydroxyethylcellulose and other cellulose derivatives, pre-gelatinized starch, starches or modified starches (including potato starch, corn starch, maize starch and rice starch) and the like. Typically glidants and lubricants may also be included in the invention. Non-limiting examples include stearic acid and pharmaceutically acceptable salts or esters thereof (for example, magnesium stearate, calcium stearate, sodium stearyl fumarate or other metallic stearate), talc, waxes (for example, microcrystalline waxes) and glycerides, light mineral oil, PEG, silica acid or a derivative or salt thereof (for example, silicates, silicon dioxide, colloidal silicon dioxide and polymers thereof, crospovidone, magnesium aluminosilicate and/or magnesium alumina metasilicate), sucrose ester of fatty acids, hydrogenated vegetable oils (for example, hydrogenated castor oil), or mixtures thereof or any other suitable lubricant. Suitably one or more binders may also be present in the invention and non-limiting examples of suitable binders are, for example, polyvinyl pyrrolidone (also known as povidone), polyethylene glycol(s), acacia, alginic acid, agar, calcium carragenan, cellulose derivatives such as ethyl cellulose, methyl cellulose, hydroxypropyl cellulose, hydroxypropyl methyl cellulose, sodium carboxymethylcellulose, dextrin, gelatin, gum arabic, guar gum, tragacanth, sodium alginate, or mixtures thereof or any other suitable binder. Suitable disintegrants may also be present in the invention. Examples include, but are not limited to, hydroxylpropyl cellulose (HPC), low density HPC, carboxymethylcellulose

(CMC), sodium CMC, calcium CMC, croscarmellose sodium; starches exemplified under examples of fillers and also carboxymethyl starch, hydroxylpropyl starch, modified starch; crystalline cellulose, sodium starch glycolate; alginic acid or a salt thereof, such as sodium alginate or their equivalents and any combination thereof.

The total moisture (water) content of the UDF must be selected to ensure appropriate stability and shelf-life for the product. Those skilled in the art are able to identify acceptable ranges depending on the form of UDF selected. Softgels are particularly sensitive to water content as water will weaken and dissolve softgel gelatin capsules. Water content is typically kept below 30% and preferably below 5% of the total mass.

In preferred embodiments, certain potential contaminants are eliminated, avoided, or present at trace levels considered acceptable for human consumption. In particular, the preferred embodiments eliminate, avoid or reduce the presence of organic solvents, pest control products, di-acetyl and ammonia.

Organic solvents: In a preferred embodiment, organic solvent used to extract the cannabinoid and/or curcumin is largely removed from the preparation before formulation in the UDF. Solvent may be removed by evaporation or other known technique. In all preferred embodiments the level of residual solvent is acceptable under ICH guideline topic Q3C(R5). The objective of this guideline is to recommend acceptable amounts for residual solvents in pharmaceuticals for the safety of the patient. The guideline recommends use of less toxic solvents and describes levels considered to be toxicologically acceptable for some residual solvents.

Trace pest control product: In a preferred embodiment any pest control product used in the cultivation of Cannabis or the curcumin, and any derivatives thereof, are removed before combination of the components in the UDF. If such pest control products cannot be fully removed, they preferably do not exceed any maximum residue limit specified for the pest control product, its components or derivatives under the Pest Control Products Act (Canada), or the corresponding act in the relevant country.

Oral formulations of the invention may be further improved by eliminating and ensuring undetectable levels of contaminants that are negatively associated with Cannabis consumption. For example, preferred embodiments of the invention comprise no detectable levels of di-acetyl (CH₃CO)₂, also called 2,3-butanedione, an additive sometimes used in preparations of Cannabis for smoking/vaping. Also preferred is no detectable level of ammonia, which may contaminate the source Cannabis crop due to over-fertilization and lack of flushing during hydroponic cultivation.

In a preferred embodiment, the UDF meets the requirements of a dissolution or disintegration test that is applicable to its formulation and that is set out in European Pharmacopoeia, The Canadian Formulary, The United States Pharmacopoeia, and/or The Pharmaceutical Codex: Principles and Practices of Pharmaceuticals.

### General UDF Production Methods

Having selected the amounts and concentrations of all ingredients of the oral formulation of the invention, the ingredients will be formulated together for preparing the unit dosage form. Those skilled in the art are familiar with identifying preferred formulation techniques for the UDF. In a preferred embodiment, the UDF is a pill, tablet, capsule, film, or wafer, any of which may optionally be orally disintegrating, or a lollipop, lozenge, oil, tincture, or syrup. The formulation process will be adjusted accordingly. Pills and tablets are prepared from solid formulations. Syrups, oils and tincture are liquid formulations. An orally disintegrating film, wafer, tablet or a lollipop or lozenge provides the UDF in an oral form wherein the active ingredients are at least partly absorbed directly in the buccal cavity. Capsules may be either solid formulations (e.g. powders or particles in a hard-gel) or liquid formulations (e.g. oil-based formulations used in soft-gels). Oil based formulations with little or no water are typically easily encapsulated. Such oil-based preparations may be mixed with a surfactant, such as but not limited to Labrasol™. Oil-in-water formulations may comprise microemulsions, liposomes, nanoemulsions and other forms known in the art.

NHP component may be physically separated from cannabinoid, or the two components may be mixed together. Physical separation by particles (which do not mix) or by capsule-within-capsule design. Mixing together can be achieved by formulation in the same liquid carrier, or by mixing of powders/particulates before capsule loading. An oil-in-water type emulsion, and other variants where the components may be separated at molecular level by hydrophilicity is considered “mixed together”, in the sense that cannabinoids and NHPs are evenly dispersed throughout the entire capsule UDF.

Preferred capsule types are soft gelatin capsules (softgels) and hard gelatin capsules. Soft Gelatin Capsules (softgels) are well known in the art. Typically soft-gels are used for formulations not based on water, such as oil-based solutions, because water based solutions would dissolve the gelatin. The basic steps of softgel manufacturing are: Gelatin Preparation (the process of blending and heating granulated gelatin into a thick syrup for use in encapsulation); Fill Material Preparation (the process of preparing the non-aqueous oil or paste containing the NHP and cannabinoid components that will be encapsulated); Encapsulation (the process of converting the gel mass into a thin layer of gelatin and wrapping it around the fill material to form a softgel); Drying (the process which removes excess moisture from the gelatin shell to shrink and firm up the softgel); the softgel could incorporate a Coating step (the process of coating the capsule with a coating designed to release the capsule within the digestive system); and finally Cleaning, Inspection and Sorting. Automated or semi-automated manufacturing of softgels and can be achieved using commercially available equipment, such as that provided by CapPlus Technologies, SaintyCo, and many others.

Hard gelatin capsules are made of two parts, the body and a cap. This form of capsule holds dry ingredients in the form of powders, granules or tiny pellets. They may also include Cannabis oils of various viscosity, such as diluted Cannabis oil and concentrated Cannabis extracts. The body is first filled with the mix of active ingredients and any excipients used, and then closed with the cap using either a manual or automated or semi-automated capsule filling machine, such as those commercially available from Bosch, Zanazzi, etc. Banding of hard gelatin capsules is sometimes useful to prevent leakage.

A wide range of capsule sizes are suitable for use with the invention. A UDF in capsule form may be any size suitable for human swallowing and for example may be selected from among any of the standard commercial capsule sizes, and/or may be selected from among about 100 mg, 200 mg, 250 mg, 300 mg, 400 mg, 500 mg, 600 mg, 700 mg, 750 mg, 800 mg, 900 mg, 1000 mg or any about 10 mg interval between 0 mg and 1000 mg.

The inventors recognize that advantages may be achieved by use of a dose form that is substantially opaque to one or both of ultraviolet and visible light, such as a photo- and/or UV-opaque gelatin capsule. A general form of this technology is described in co-owned patent application U.S. Ser. No. 62/837,848 filed 24 Apr. 2019 and incorporated herein by reference.

Delayed release to the gastrointestinal track can be achieved for softgels or hard gels by enteric coatings which delay disintegration until after passing from stomach to the intestine; or by formulation techniques such as pellets which resist release until they pass into a specific intestinal domain. Such techniques are widely known in the art. An example is WIPO patent publication WO2017075215A1 to McGuffy and Bell for extended release film-coated softgel or hard-shell capsules.

A wide variety of technologies are available for a buccal or sublingual formulation such as an orally disintegrating thin film, wafer or tablet, or a lollipop, and/or lozenge. Sublingual tablets, wafers, films and strips can be designed to rapidly disintegrate (5-15 seconds) providing rapid access to buccal cavity capillaries and avoid the hostile environment of the gastrointestinal track. Lollipops and lozenges provide a combination of buccal and gastric administration. The technologies are widely used with therapeutic agents where rapid onset is desired. (See Lamey and Lewis “Buccal and Sublingual Delivery of Drugs” Ch 2 in “Routes of Drug Administration” Ed. Florence and Salole (Butterworth-Heinemann)).

Association with Signifier

The UDF of the invention preferably comprises a signifier which allows the consumer to determine the defined dose of selected cannabinoids therein. A “signifier” means a mark, symbol, indicia, striation or the like which may be perceived visually or by touch, which provides information to a consumer about the UDF's specific defined dose. The signifier chosen may have elements of meaning, such as a number and unit, (e.g. “5 mg” or “10 mg” or simply “5” or “10”) or it may be an abstract signifier, where its meaning, in terms of defined dose, can be determined by reference to a standard. The meaning may be determined directly by the consumer or indirectly via a device.

The signifier may be associated directly with the UDF after encapsulation by such means as embossing, or by colour, pattern or shape feature. Alternatively, the signifier may be associated with the packaging. The packaging may include signifiers directly interpreted by consumers or signifiers which are machine readable codes. In all embodiments, the signifier allows the consumer to determine the defined dose of selected cannabinoid(s) therein and optionally the dose of the NHP and any other constituent.

The signifier may be associated directly with the UDF before, during or after encapsulation by such means as edible ink(s) imprinted on the surface of the capsule, or by embossing, by engraving (such as laser-engraving), or by color, pattern or shape feature. The edible ink applied to the capsule may include shellac from about 10% to about 30% by weight, about 20% to 70% by weight of at least one solvent, and at least one soluble or insoluble pigment from about 10% wt to about 40% wt. The shellac provides structure, enhances adherence to the printing plate and capsule, and acts as a pigment carrier. An edible ink formulation may include 10% wt to about 30% wt shellac.

### Packaging

After a UDF is manufactured, storage and delivery to consumer may be provided by:
a. Packaging the UDF individually in a blister pack; or
b. Packaging multiple UDFs in a re-sealable package.

The UDF is preferably provided in a sealed package, which functions as a barrier limiting moisture fluctuation, reducing oxidation, and enhancing shelf-life, etc. The packaging is optionally a gas-impermeable container having a hermetic closure which in the context of the present invention includes a blister pack. The UDFs may be individually sealed and packaged in blister packs. The blister packs may be designed to be child resistant and/or senior friendly in order to increase safety and convenience. While physically protecting the matrix units, the blister pack controls humidity and is impermeable to gas exchange thereby enhancing shelf life.

Examples of the substantially gas exchange impermeable packaging include, but are not limited to, A1/A1 blister, and A1-polychloro-3-fluoroethylene homopolymer/PVC laminate blister. Alternatively, the sealed package may be a re-sealable multi-package impermeable to gas exchange.

UDFs of the invention may be expelled from production into the open blister cavities. Cavity depth and shape must be suitable for the unit. The open blister cavity is then sealed with a gas impermeable membrane to maintain quality of product and to reduce dehydration, rehydration or oxidation. To eliminate oxidation altogether, the packaging may be performed in an inert gas atmosphere. Optionally the blister is packed in an inert gas atmosphere such as nitrogen gas comprising little or no oxygen. To achieve this objective, the final sealing step of the packaging method may be operated in the inert gas atmosphere in a gas enclosure protected from ambient air.

### EXAMPLES

### Example 1: Bioinformatics

The inventors have employed a variety of bioinformatics tools to identify the biochemical synergies of the oral combinations proposed herein and to predict their therapeutic effects.

### Bioinformatic Tools

Gene Expression Overlaps: Gene expression overlaps were identified from the Comparative Toxicogenomics Database (CTD), MDI Biological Laboratory, Salisbury Cove, Maine, and NC State University, Raleigh, N.C. World Wide Web (URL: http://ctdbase.org/). (see Davis A P et al. The Comparative Toxicogenomics Database: update 2017. Nucleic Acids Res. 2016 Sep. 19. Genevenn and Enrichr programs have also been employed. Genevenn (http://genevenn.sourceforge.net) finds gene expression overlaps. Enrichr (http://amp.pharm.mssm.edu/Enrichr/) was applied to the gene overlap to search through libraries of data (disease associations, expression data, biochemical databases, etc) that matches the gene/protein to the overlapping pathways and that way identified the gene expression pathways. Enrichr identified cell signaling pathways for the overlapping genes. Enrichr is named for the function of the gene lists/terms that are enriched cell lines that express the receptors.

NHP-Protein Interactions: NHP-protein interactions are taken from STITCH (search tool for interactions of chemicals'), a bioinformatics tool available at http://stitch.embl.de. STITCH lists known chemical-protein interactions and integrates information about interactions from metabolic pathways, crystal structures, binding experiments and drug-target relationships. (Sklarczyk et al. (2015) STITCH 5: augmenting protein-chemical interaction networks with tissue and affinity data. NAR 2016 (44) D380-D384.) STITCH has been used by the inventors to investigate the shared pathways activated by the individual product components.

### Known Metabolic Pathways of Curcumin

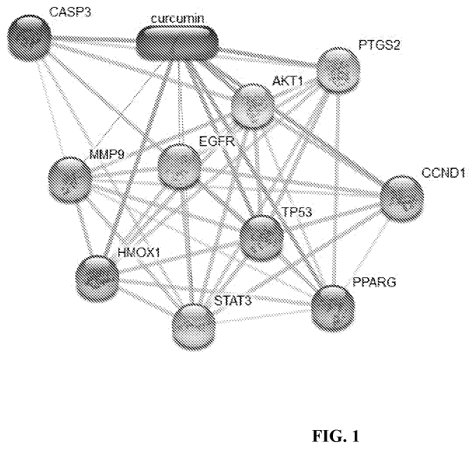

At a biochemical level, curcumin directly interacts with human metabolic pathways illustrated in FIG. 1 (adapted from STITCH) and listed in Table 2.

TABLE 2
Predicted Functional Partners of Curcumin

| Acronym | Full Name | Score |
|---|---|---|
| EGFR | epidermal growth factor receptor | 0.987 |
| CCND1 | cyclin d1 | 0.967 |
| AKT1 | v-akt murine thyoma viral oncogene homolog 1 | 0.967 |
| HMOX1 | heme oxygenase (decycling) 1 | 0.966 |
| PTGS2 | prostaglandin-endoperoxide synthase 2 | 0.964 |
| TP53 | tumor protein p53 | 0.962 |
| STAT3 | signal transducer and activator of transcription 3 | 0.959 |
| | (acute-phase response factor) | |
| CASP3 | caspase 3 | 0.959 |
| PPARG | peroxisome proliferator-activated receptor gamma | 0.957 |
| MMP9 | matrix metallopeptidase 9 | 0.956 |

Using bioinformatic tools, the inventors have now identified selected cannabinoids where effects on shared underlying pathways not previously recognized lead to the synergistic and surprising results of the invention.

### Predicted Therapeutic Effects Based on Shared Metabolic and Gene Expression Pathways

### Example 1A: Curcumin and THC

The gene and protein interaction, gene expression signaling pathways and the anticipated clinical indications for the synergistic therapeutic application of Curcumin and THC combination was carried using CTD, STITCH (Szklarczyk et al 2015), Genevenn and Enrichr programs. Gene interactions between Curcumin and THC identified 10.29% gene overlap (192 genes). See FIG. 2. The main signaling pathways are gene networks regulating gene expression in response to a variety of stimuli, including cytokines, growth factors, stress, and bacterial and viral infections. The inventors have identified that specific gene expression pathways activated by both compounds include the AP-1 transcription factor network, Glucocorticoid receptor (GR) regulatory network, ATF-2 transcription factor network and the NF-kB signaling network. Based on these observations, certain predictions as to therapeutic efficacy may be drawn.

The NF-κB signaling network is the main pathway that plays a crucial role influencing a broad range of biological processes including innate and adaptive immunity, inflammation and stress responses. This pathway can be activated by infection, stress, diet, chemotherapy, obesity, addiction as well as by LPS and variety of pro-inflammatory cytokines such as IL-1(3, TNF-α, IL-6, IL-8. Following stimulation, NF-κB transcription factor increases the expressions of genes, production of cytokines, enzymes and adhesion molecules.

Based on the observed overlap for the AP-1, ATF-2 and NF-κB signaling pathways the anticipated synergistic therapeutic effect for Curcumin-THC combination is expected in the treatment of neuropathic pain, inflammatory pain, chronic pain, acute and chronic inflammation as well as cachexia, anorexia, fatigue, depression, anxiety, cognitive impairment and sleep disorders.

The glucocorticoid (GR) regulatory network is the main gene expression pathway expressed in almost every cell in the body that regulates genes controlling the development, metabolism, immune response and stress response. Targeting this pathway with Curcumin-THC combination is beneficial in the treatment of anxiety and stress-related disorders such as mental health disorders, obsessive-compulsive disorder, PTSD-associated insomnia, nightmares and PTSD symptoms.

### Example 1B: Curcumin and CBD

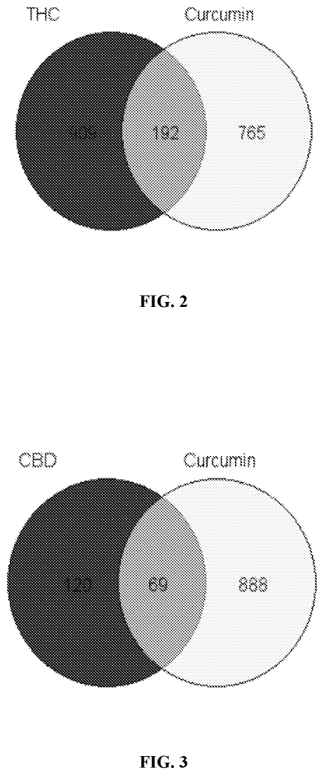

Gene interactions between Curcumin and CBD identified 6.4% gene overlap (69 genes). See FIG. 3. The signaling pathways were mainly gene networks regulating gene expression in response to a variety of stimuli, including cytokines, growth factors, stress, and bacterial and viral infections. Specific gene expression pathways were ATF-2 transcription factor network, IL-23-mediated signaling network, Glucocorticoid receptor (GR) regulatory network, AP-1 transcription factor network, IL-12-mediated signaling network and the NF-kB signaling network.

Based on the observed overlap for the ATF-2, AP-1, IL-23, IL-12 and NF-κB transcription factors signaling pathways the synergistic therapeutic effect for Curcumin-CBD combination is especially preferred for use in the treatment of neuropathic pain, inflammatory pain, chronic pain, acute and chronic inflammation as well as cachexia, anorexia, fatigue, depression, anxiety, cognitive impairment and sleep disorders.

Based on the observed overlap in the GR regulatory pathway treatment with the combination of Curcumin-CBD is also preferred in the treatment of anxiety and stress-related disorders such as mental health disorders, obsessive-compulsive disorder, PTSD-associated insomnia, nightmares and PTSD symptoms.

### Example 1C: Curcumin and Other Cannabinoids

The Curcumin-CBDA gene expression overlap shares one common gene PTGS2 (Prostaglandin-Endoperoxide Synthase 2) (identified by STITCH analysis). The PTGS2 also known as cyclooxygenase-2 (COX-2) represent important target since CBDA has been shown to selectively inhibit COX-2 activity with an IC50 value (50% inhibition concentration) at −2 μM, having 9-fold higher selectivity than COX-1 inhibition (Takeda et al 2008). Curcumin has been shown to prevent COX-2 protein phosphorylation (Patel et al 2008) and the curcumin main active ingredients such as demethoxycurcumin and bisdemethoxycurcumin were attributed to the inhibition of COX-2 expression (Guo et al 2008). COX-2 cyclooxygenase is a target for NSAIDs and COX-2 specific inhibitors and is expressed by cells that are involved in inflammation. COX-2 is primarily responsible for the synthesis of prostanoids involved in acute and chronic inflammatory states of pathological processes (DeWitt, 1999; Hinz and Brune, 2002).

CBDA is a TRPA1 agonist (De Petrocellis et al 2008), TRPV1 agonist (Ligresti et al 2006), and TRPM8 antagonist (De Petrocellis et al 2008) which may also reflect its potential as an analgesic. It is also anti-inflammatory (Izzo et al 2009, Ruhaak et al 2011, Takeda et al 2008) via selective COX-2 inhibition (Rock et al 2016, Takeda et al 2008), and has anti-nausea properties (Rock et al 2016, Bolognini et al 2013). TRPA1 is a receptor target common to both Curcumin and CBDA binding.

THCA is a TRPA1 partial agonist and TRPM8 antagonist (De Petrocellis et al 2008) which underlies a role in analgesia and has been shown to have anti-inflammatory (Ruhaak et al 2011) and anti-nausea properties (Rock et al 2013). As such, TRPA1 is a receptor target common to both Curcumin and THCA binding which implicates this combination in analgesic and anti-inflammatory indications. THCA is also identified as a PPAR gamma agonist that is useful for treatment of diet induced obesity, diabetes, weight gain and metabolic syndrome. THCA has been also shown to have anti-inflammatory (Ruhaak et al 2011) and anti-nausea properties (Rock et al 2003). Both GABA and the Cannabinoid target the same CB1 receptor with moderate binding at Ki affinity binding ranging from 760-1,000 nM (Stefano et al, 1997).

### Example 2: In Vitro Biological Examples

Amounts and concentrations of curcumin and the selected cannabinoid for testing in the assays below (both in vitro and in vivo) are chosen to correspond to the dose that would be expected upon administration to a human of the oral formulation or one or more unit dosage forms of the invention taken at the same time. For example, in cell-based assays the amounts are adjusted to correspond to present the cells with the expected physiological level that would be encountered in a human consuming an oral formulation of the invention. Similarly, in animal models, the amount tested is adjusted from the UDF used in humans to a corresponding ratio in the animal based on mg/kg, factoring in expected oral uptake and absorption differences. Those skilled in the art are familiar with defining and selecting the concentrations to be tested in the assays and extrapolating back to the appropriate dosage for the UDF in humans.

Background for Identification of Synergies from Cell-Based and Biochemical Assays

### Synergy Index

The use of multiple therapeutic agents may target multiple targets and/or multiple diseases simultaneously. The use of agents with similar mechanisms or modes of action may also maximize the effect against single target or a disease and treat it more effectively. In addition, coordinated action at multiple molecular targets can provide unique therapeutic benefit not achievable with the “one-drug, one-target” paradigm.

Two or more therapeutic agents that individually produce overtly similar effects will sometimes display greatly enhanced effects when given in combination. When the combined effect is greater than that predicted by their individual potencies, the combination is described as synergistic, and more specifically a positive synergy. A synergistic interaction allows the use of lower sub-therapeutic doses of the combination constituents, a situation that may reduce potential adverse reactions. Sometimes a positive synergy may conceptually be stated as a “1+1=3” effect.

The possible favorable outcomes for synergism include, but are not limited to:
a. Increasing the efficacy of the beneficial therapeutic effect
b. Decreasing the dosage but increasing or maintaining the same efficacy to reduce cost and avoid undesirable adverse effects
c. Minimizing or slowing down the development of drug resistance, and
d. Providing selective synergism against target (or efficacy synergism) versus host (or toxicity antagonism)

The contrary outcome, a negative synergy, may be observed for certain combinations. A negative synergy would be observed where a second compound competitively inhibits the activity of the first compound. The mechanism of inhibition is not essential to the resulting effect, but such inhibition may be direct (antagonistic) at the active site of relevant enzymes, it may be allosteric (acting through another site on an enzyme) or it may be through a different enzyme in a linked pathway. Negative synergies may be described as a “1+1=½” effect, and include but are not limited to:
a. Reducing efficacy that would otherwise be expected were the components used alone.
b. Increasing the dosage required to maintain the same efficacy;
c. Reducing unwanted effects of one component found in a mixture of compounds.
   i. For example, negative synergy can reduce or eliminate the psychoactive effect of THC from a mixture of cannabinoids containing THC. This would allow the consumer to benefit from the non-psychoactive cannabinoids of the formulation, and the non-psychoactive effects of THC, without experiencing the psychoactive “high”.).

Evaluation of synergistic effects for cannabinoid and NHP combinations can be evaluated in cell based and biochemical receptor binding assays, by determining effects over a range of ratios and concentrations and analyzed by CalcuSyn software program (Biosoft, Ferguson, Mo., USA). This program could be used for dose effect analysis for single agents using the median-effect equation and for agents in combination using both the median-effect equation and the combination index equation (Chou and Talalay, 1984, Chou and Hayball, 1996, Chou and Martin, 2005 and Chou, 2006). The occurrence of ratio-dependent synergy is determined by plotting the combination index (CI<1, synergy (or positive synergy); CI˜1, additivity; and CI>1, antagonism (or negative synergy) versus the fraction of cells affected (Fa), which indirectly reflects the therapeutic agent concentration.

### In Vitro Cell-Based Assays

### 1. NF-κB Luciferase Assay

NF-κB (Nuclear Factor-Kappa B) signaling network is the main pathway that plays a crucial role influencing a broad range of biological processes including innate and adaptive immunity, inflammation and stress responses. It could be activated by infection, stress, diet, chemotherapy, obesity, addiction as well as by a variety of pro-inflammatory cytokines such as IL-1β, TNF-α, IL-6, IL-8.

The NF-kB pathway inhibition in response to treatment with the Cannabinoid and NHP either as single agents or in combination will be monitored using the Luciferase Reporter gene assay described in Del Prete et al, J. Nat. Prod. 2017, 80, 2276-2283.

For the anti-NF-κB activity stably transfected NIH-3T3-KBFLuc cells are treated with different concentration of NHP compounds for 30 minutes and then stimulated with TNF-α (30 ng/mL). After treatment, the cells are washed twice in phosphate-buffered saline and lysed in 25 mM Tris-phosphate pH 7.8, 8 mM MgCl2, 1 mM DTT, 1% Triton X-100, and 7% glycerol during 15 min at room temperature in a horizontal shaker. Luciferase activity in the supernatant is measured using a TriStar2 Berthold/LB942 multimode reader (Berthold Technologies) following the manufacturer's instructions of the luciferase assay kit (Promega, Madison, Wis., US). For NF-κB inhibition the RLU (relative light units) is calculated, and the results are normalized to 100% stimulation induced by induced by TNF-α (100% activation). The results are presented from three independent experiments.

### 2. CB₁R Cannabinoid Receptor Agonist and Antagonist Assays

The CB₁R and CB₂R agonistic and antagonistic activities in response to treatment with the Cannabinoid and NHP either as single agents or in combination are measured using the HEK293T-CB₁R and HEK293T-CB₂R cells lines stably transfected with human CB1R and CB2R cDNA respectively. Briefly, HEK293T-CB1 cells are transiently transfected with 0.2 μg of the reporter plasmid CRE-luc that contains six consensus cAMP responsive elements (CRE) linked to firefly luciferase reporter gene using Roti-Fect (Carl Roth, Karlsruhe, Germany) following manufacturer's instructions. The increase in cAMP levels activates the pCRE-Luc system, inducing the expression of the luciferase reporter gene.

For CB1R agonistic activity, the transfected HEK293T0CB1-CRE-luc cells are treated with a range of concentrations of the compounds. For CB1R antagonistic and allosterism activity, these cells are incubated with different concentrations of the compounds for 30 minutes and then treated with the CB1R agonist CP-55940. Forskolin, an adenylate cyclase activator, is used at 10 μM along as a positive control of cAMP signaling pathway activated by a CB1R-independent mechanism; Cp-55940, a CB1R agonist, is used at 1 μM alone as a positive control of cAMP signaling pathway activated by a CB1R-dependent mechanism.

After 6 hours of stimulation the cells are washed twice in phosphate-buffered saline and lysed in 25 mM Tris-phosphate pH 7.8, 8 mM MgCl2, 1 mM DTT, 1% Triton X-100, and 7% glycerol during 15 min at room temperature in a horizontal shaker. After centrifugation, luciferase activity in the supernatant is measured using a TriStar2 Berthold/LB942 multimode reader (Berthold Technologies) following the instructions of the luciferase assay kit (Promega, Madison, Wis., USA). The results are represented as the mean of at least five independent experiments, with the SD lower than 15%.

### 3. CB2R Cannabinoid Receptor Agonist Assays

For CB2R agnostic activity HEK293T-CB2-CRE-luc cells are treated either with different concentrations of the compounds or with 1 μM WIN-55,212-2 (CBR2 agonist) for 30 minutes and then with 10 μM Forskolin. Forskolin, an adenylate cyclase activator, is used at 10 μM along as a positive control of cAMP signaling pathway activated by a CB1R-independent mechanism; Win-55,212-2, a CB2R agonist, in the presence of 10 μM forskolin, is used at 1 μM as a positive control of CB2R agonistic activity.

After 6 hours of stimulation the cells are washed twice in phosphate-buffered saline and lysed in 25 mM Tris-phosphate pH 7.8, 8 mM MgCl2, 1 mM DTT, 1% Triton X-100, and 7% glycerol during 15 min at room temperature in a horizontal shaker. After centrifugation, luciferase activity in the supernatant is measured using a TriStar2 Berthold/LB942 multimode reader (Berthold Technologies) following the instructions of the luciferase assay kit (Promega, Madison, Wis., USA). The results are represented as the mean of at least five independent experiments, with the SD lower than 15%.

Thus, as part of the Synergy Index, the combinations of the invention, and the components separately, can be tested for agonist and antagonist behaviour against CB₁R and agonist behaviour against CB₂R. Such assays are widely available commercially. A preferred assay is the Ready-to-Assay™ CB₁R (catalog: HTS019RTA) and Ready-to-Assay™ CB₂R (catalog: HTS020RTA) cell-based assays provided by Millipore.

The combinations of the invention are found to demonstrate surprising and unexpected synergies.

### Example 2A: Curcumin Inhibits CB1R Agonism by THC (=Negative Synergy) (CB1R Agonism Assay)

The agonistic activity of curcumin alone or in combination with THC or THCA on CB1 receptor was analyzed using the HEK293T-CB1 cell line and the CRE-Luc reporter that is sensitive to the increase of cellular cAMP level after CB1R ligand occupation. The CB1R agonistic activity was reflected by the induction of CRE-Luc activity relative and expressed as a fold change over non-stimulated cells.

95% pure crystalline powder curcumin (Enzo Life Sciences, #ALX-350-028), dissolved in dimethyl sulfoxide (DMSO), was prepared to five different concentrations alone (0.1, 1, 5, 10 and 15 μM) or in the presence of 10 μM THC or 10 μM THCA dissolved in DMSO and were evaluated on CB1R agonistic activity. Table 3 summarizes obtained data.

TABLE 3
Curcumin alone and curcumin combinations CB1R agonistic activity data. Data are represented
as fold change of CB1R activity. An increase of the fold change over 2.5-fold indicates CB1R
agonistic activity. Data are from independent experiments where the positive control
(CP55940; CB1R agonist) showed an induction of CB1R activity ≥2.5-fold increase. Only
treatments that the mean of three consecutive independent experiments shows a positive
result (CB1R activity ≥2.5-fold increase) are considered agonists of CB1R activity.

| | | | | Conclusions: Is |
|---|---|---|---|---|
| | Experiment 1 | Experiment 2 | Experiment 3 | CB1R agonist |
| | Fold change | Fold change | Fold change | effect observed? |
| CP 2,5 μM (control agonist) | 2.5 | 2.5 | 2.5 | + |
| Curcumin 0.1 μM | 1.0 | 1.3 | 1.1 | Negative |
| Curcumin 1 μM | 1.0 | 1.1 | 0.8 | Negative |
| Curcumin 5 μM | 1.0 | 1.1 | 0.7 | Negative |
| Curcumin 10 μM | 0.9 | 0.8 | 0.9 | Negative |
| Curcumin 15 μM | 0.6 | 0.8 | 1.6 | Negative |
| THCA 10 μM | 0.9 | 0.8 | 1.1 | Negative |
| Curcumin 0.1 μM + THCA 10 μM | 0.7 | 0.8 | 1.4 | Negative |
| Curcumin 1 μM + THCA 10 μM | 1.2 | 0.8 | 1.0 | Negative |
| Curcumin 5 μM + THCA 10 μM | 0.8 | 0.8 | 0.8 | Negative |
| Curcumin 10 μM + THCA 10 μM | 0.7 | 0.6 | 0.9 | Negative |
| Curcumin 15 μM + THCA 10 μM | 0.6 | 0.8 | 0.6 | Negative |
| THC 10 μM | 4.5 | 3.4 | 2.7 | + |
| Curcumin 0.1 μM + THC 10 μM | 4.8 | 3.6 | 2.5 | + |
| Curcumin 1 μM + THC 10 μM | 4.1 | 3.7 | 3.3 | + |
| Curcumin 5 μM + THC 10 μM | 2.2 | 2.0 | 1.7 | Reduced agonist |
| | | | | effect |
| Curcumin 10 μM + THC 10 μM | 1.2 | 1.3 | 1.0 | Eliminated |
| | | | | agonist effect |
| Curcumin 15 μM + THC 10 μM | 1.1 | 0.2 | 0.9 | Eliminated |
| | | | | agonist effect |

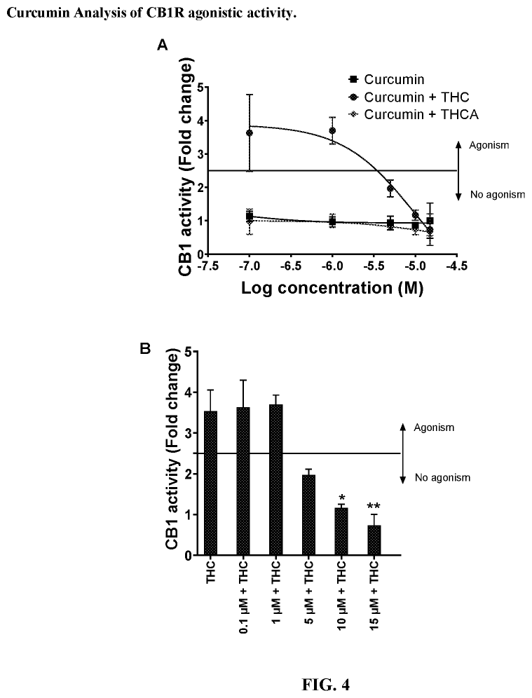

Curcumin alone and Curcumin-THCA combination did not show CB1R agonistic activity in any tested concentration. However, in the curcumin-THC combination, the expected THC-associated CB1 agonistic activity was progressively reduced as the concentration of curcumin was increased being completely abolished at the highest tested curcumin concentration (FIG. 4A). The IC50 value of curcumin to block the 10 μM THC-associated CB1R agonistic activity was 2,68±0,63 μM. An additional analysis comparing the effects of THC alone versus curcumin-THC treatments showed a statistically significant difference at 10 and 15 μM confirming the curcumin blocking action of the THC CB1R agonistic activity (FIG. 4B). This dose-dependent effect confirmed the orthosteric CB1 antagonistic activity of curcumin.

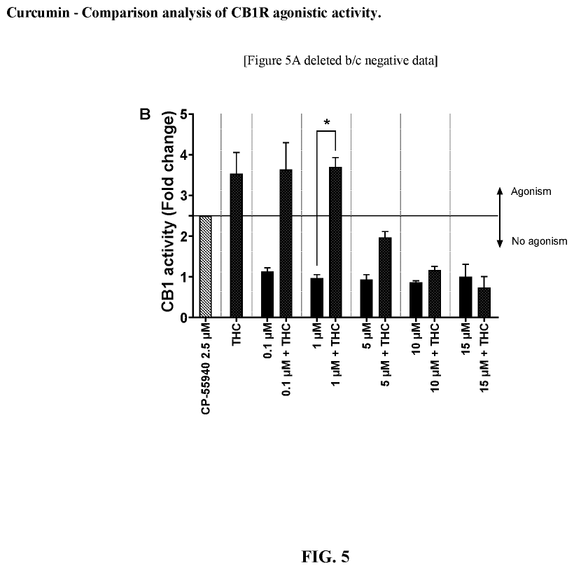

A deeper comparison study of the treatments was performed to further analyze the possible differences between curcumin alone and curcumin combinations. This analysis showed statistically significant different effects between curcumin alone and curcumin-THC combination treatment. This difference, caused by the THC-associated CB1 agonistic activity, disappeared at highest curcumin concentrations because the agonist effect of THC was completely inhibited by the antagonist action of curcumin (FIG. 5).

### Example 2B: Curcumin Inhibits CB1R Activation by THC (CB1R Antagonism Assay)

The antagonistic activity of curcumin alone or in combination with THC, CBD or THCA on CB1 receptor was further analyzed in a CB1R antagonism assay using the HEK293T-CB1 cell line and the CRE-Luc reporter that is sensitive to the increase of cellular cAMP level after CB1R ligand occupation. The CB1R antagonistic activity was reflected by the inhibition percentage of CP55940-induced CRE-Luc activity, considered the 100% of stimulation, as described in experimental design section.

As in Example 2A, five different concentrations of curcumin alone (0.1, 1, 5, 10 and 15 μM) or in the presence of 10 μM THC or THCA or, additionally in this case, 1 μM CBD were evaluated on CB1R antagonistic activity. Table 4 summarizes obtained data.

TABLE 4
Curcumin alone and curcumin combinations CB1R antagonistic activity data. Data are represented
as percentage of CB1R activity. A reduction of the percentage below 50% indicates CB1R
antagonistic activity. Data are from independent experiments where the positive control
(CP55940; CB1R agonist) showed an induction of CB1R activity ≥2.5-fold increase. Only
treatments that the mean of three consecutive independent experiments shows a positive
result (CB1R activity ≤50%) are considered CB1R antagonists.

| | | | | Conclusions: Is |
|---|---|---|---|---|
| | Experiment 1 | Experiment 2 | Experiment 3 | CB1R antagonist |
| | % of activity | % of activity | % of activity | effect observed? |
| CP 2,5 μM (control non-antagonist) | 100.0 | 100.0 | 100.0 | Negative |
| Curcumin 0.1 μM | 125.9 | 112.3 | 110.3 | Negative |
| Curcumin 1 μM | 109.6 | 122.5 | 128.0 | Negative |
| Curcumin 5 μM | 20.0 | 89.0 | 50.9 | Mild |
| Curcumin 10 μM | 24.1 | 38.1 | 27.0 | + |
| Curcumin 15 μM | 15.2 | 14.0 | 7.0 | + |
| THCA 10 μM | 116.7 | 116.0 | 56.4 | Negative |
| Curcumin 0.1 μM + THCA 10 μM | 117.1 | 166.5 | 75.1 | Negative |
| Curcumin 5 μM + THCA 10 μM | 22.1 | 64.7 | 47.7 | + |
| Curcumin 10 μM + THCA 10 μM | 35.4 | 37.6 | 18.5 | + |
| Curcumin 15 μM + THCA 10 μM | −2.4 | −9.7 | −0.3 | + |
| CBD 1 μM | 61.3 | 55.9 | 57.4 | Negative |
| Curcumin 0.1 μM + CBD 1 μM | 67.6 | 58.2 | 56.3 | Negative |
| Curcumin 1 μM + CBD 1 μM | 115.4 | 106.6 | 92.7 | Negative |
| Curcumin 5 μM + CBD 1 μM | 85.7 | 67.6 | 48.5 | Negative |
| Curcumin 10 μM + CBD 1 μM | 36.3 | 33.4 | 8.5 | + |
| Curcumin 15 μM + CBD 1 μM | 10.5 | −1.3 | −7.9 | + |
| THC 10 μM | 181.6 | 135.5 | 107.0 | Negative |
| Curcumin 0.1 μM + THC 10 μM | 192.2 | 142.3 | 101.8 | Negative |
| Curcumin 1 μM + THC 10 μM | 165.3 | 149.3 | 131.3 | Negative |
| Curcumin 5 μM + THC 10 μM | 87.4 | 81.9 | 66.4 | Negative |
| Curcumin 10 μM + THC 10 μM | 49.6 | 52.7 | 40.0 | + |
| Curcumin 15 μM + THC 10 μM | 45.8 | 9.7 | 36.6 | + |

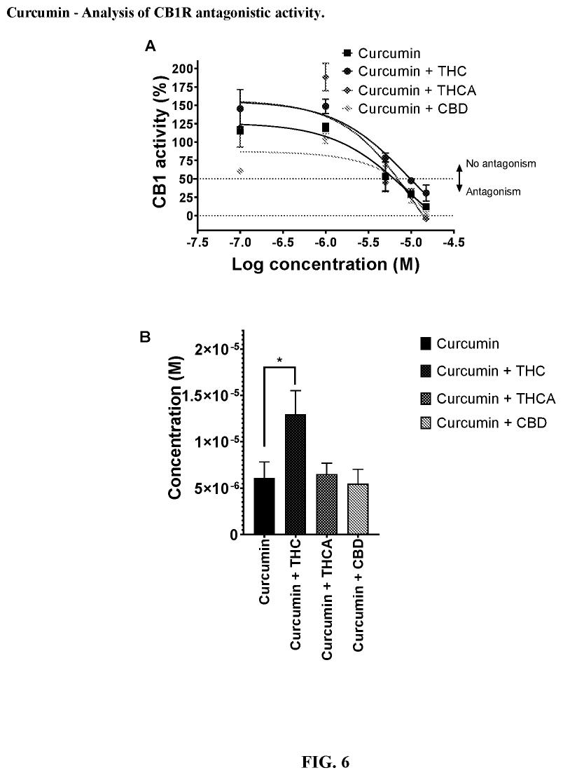

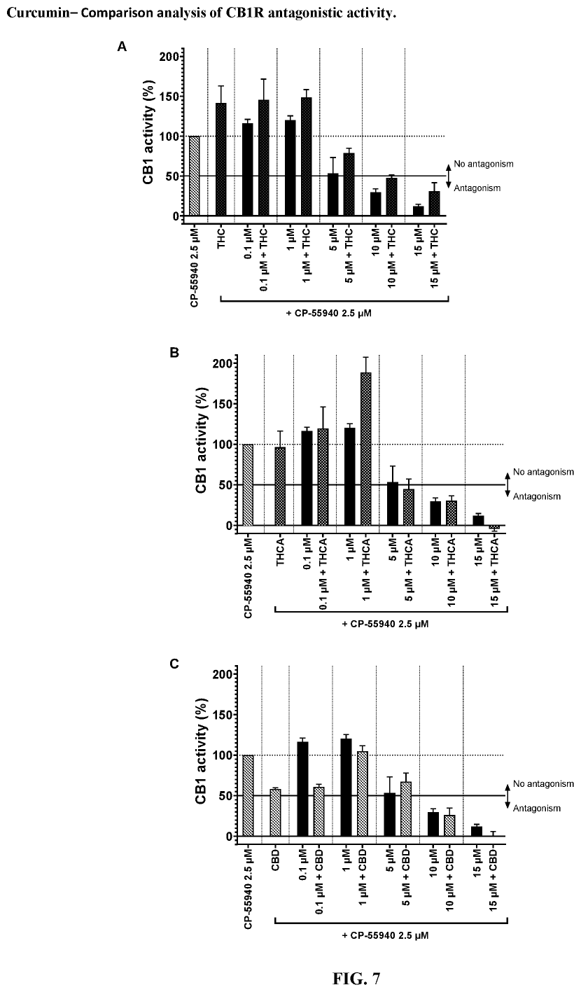

The CB1R antagonistic activity of curcumin was clear in all tested treatments (FIG. 6A). Curcumin alone CB1R antagonistic activity IC50 value was 6,18±1,71 μM. Curcumin-THCA and curcumin-CBD combinations showed CB1R antagonism, but these were not statistically significant versus curcumin alone (Curcumin-THCA IC50=6,52±1,87 μM and Curcumin-CBD IC50=5,47±1,55 μM). However, curcumin-THC combination treatment showed a statistically significant reduction of the curcumin CB1R antagonism activity with the consequent increase of the IC50 (*, p<0.05. Curcumin-THC IC50=12,97±2,54 μM) (FIG. 6B).

A deeper comparison study of the treatments was performed to further analyze the possible difference between curcumin and curcumin combination treatments. The curcumin-THC combination showed a statistically significant increase of the IC50 value (FIG. 6A). The comparison effects between individual tested concentrations showed a declining trend (FIG. 7A). Finally, the CBD-curcumin combination analysis revealed that at 0.1 μM curcumin prevailed over the CBD negative allosteric activity on CP-55940 effect; however, from the following curcumin concentration (1 μM), the CBD-induced antagonist activity was blocked. From this point the antagonist activity of curcumin started to prevail and the combination with CBD did not improve the antagonist curcumin effect (FIG. 7C).

### Example 2C: Curcumin is an Agonist of the CB2 Receptor Both Alone and in Combination with Cannabinoid (THC and THCA)

The agonistic activity of curcumin alone or in combination with THC or THCA on CB2R was analyzed using the HEK293T-CB2 cell line and the CRE-Luc reporter that is sensitive to the increase of cellular cAMP level after CB2R ligand occupation. WIN-55,212-2, a CB2R agonist, in the presence of 10 μM forskolin, was used at 1 μM as positive control of CB2R agonistic activity; Forskolin, an adenylate cyclase activator, was used at 10 μM alone as a positive control of cAMP signaling pathway activated by a CB2R-independent mechanism and cells without stimulus were used as negative control. Results are normalized to 100% stimulation induced by forskolin, expressed as the percentage of the CB2R activity.

As in Example 2A, five different concentrations of curcumin alone (0.1, 1, 5, 10 and 15 μM) or in the presence of 10 μM THC or 10 μM THCA were evaluated on CB2R agonistic activity. Table 5 summarizes obtained data.

TABLE 5
Curcumin alone and curcumin combinations CB2R agonistic activity data. Data are represented as
percentage of activity. A reduction of the percentage below 50% indicates CB2R agonistic activity. Data are from
independent experiments where the positive control (WIN-55,212-2, CB2R agonist) reduced the activity below
50%. Only treatments that the mean of three consecutive independent experiments shows a positive result (activity
≤50%) are considered agonistic of CB2R activity.

| | | | | Conclusions: Is CB2R |
|---|---|---|---|---|
| | Experiment 1 | Experiment 2 | Experiment 3 | agonist effect |
| | % of activity | % of activity | % of activity | observed? |
| Forskolin 1 μM (control without | 100.0 | 100.0 | 100.0 | Negative |
| agonist) | | | | |
| Curcumin 0.1 μM | 103.3 | 134.8 | 112.8 | Negative |
| Curcumin 1 μM | 103.7 | 101.3 | 117.6 | Negative |
| Curcumin 5 μM | 55.4 | 106.9 | 97.5 | Negative |
| Curcumin 10 μM | 8.1 | 15.2 | 19.8 | + |
| Curcumin 15 μM | −7.3 | 8.8 | −12.8 | + |
| THC 10 μM | 54.7 | 73.7 | 60.3 | Negative |
| Curcumin 0.1 μM + THC 10 μM | 67.7 | 75.3 | 51.6 | Negative |
| Curcumin 1 μM + THC 10 μM | 73.4 | 73.0 | 61.7 | Negative |
| Curcumin 5 μM + THC 10 μM | 52.5 | 73.2 | 76.0 | Negative |
| Curcumin 10 μM + THC 10 μM | 5.9 | 10.4 | 2.7 | + |
| Curcumin 15 μM + THC 10 μM | −4.2 | 22.0 | −5.2 | + |
| THCA 10 μM | 115.6 | 105.6 | 124.6 | Negative |
| Curcumin 0.1 μM + THCA 10 μM | 92.5 | 113.6 | 85.1 | Negative |
| Curcumin 1 μM + THCA 10 μM | 107.0 | 113.5 | 110.0 | Negative |
| Curcumin 5 μM + THCA 10 μM | 68.6 | 77.5 | 79.7 | Negative |
| Curcumin 10 μM + THCA 10 μM | 5.7 | 15.4 | 16.4 | + |
| Curcumin 15 μM + THCA 10 μM | −8.1 | −9.6 | −15.2 | + |

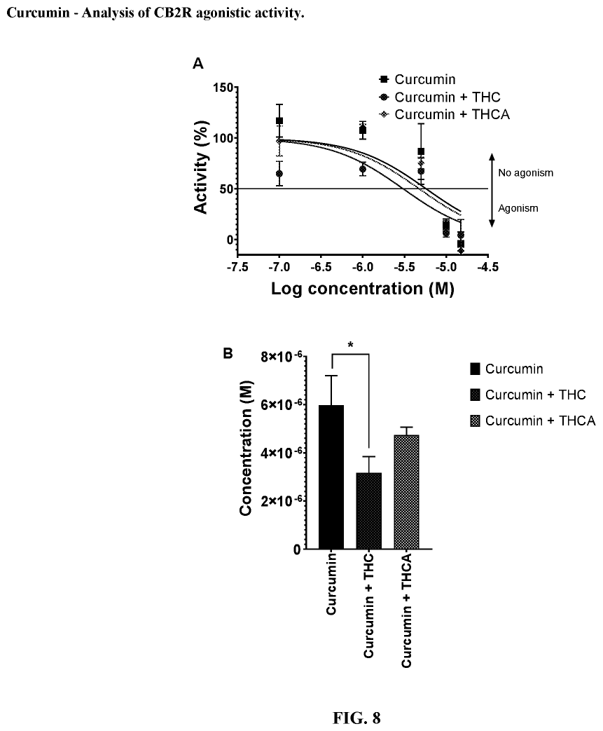

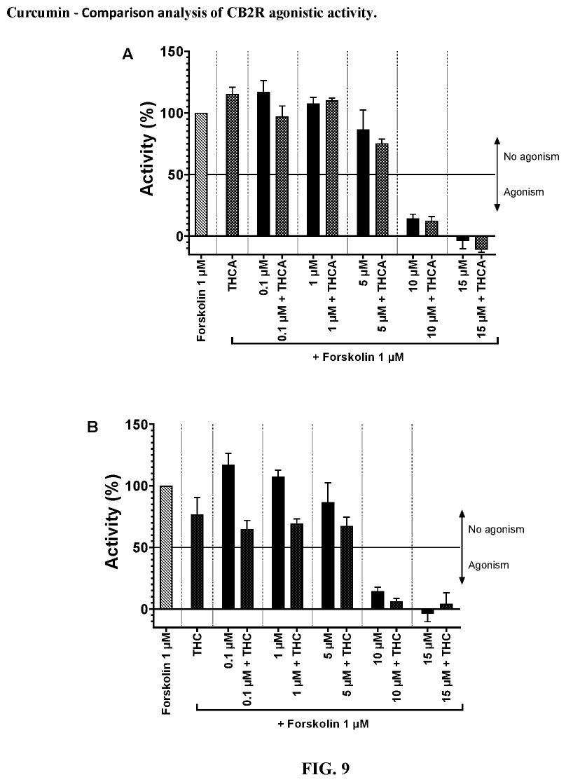

Table 5, FIG. 8 and FIG. 9 show that curcumin acts as a CB2R agonist. To determine the half maximal effective concentration (EC50): the resulting dose-response curve was plotted in nonlinear regression log [antagonist] (M) vs normalized response (%). Curcumin CB2R agonistic activity EC50 value was 5,968±1,233 μM (FIGS. 8A and 8B). The agonistic activity of curcumin was slightly increased when it was combined with THC, causing a reduction of the EC50 (*, p<0.05. 3,169±0,677 μM) (FIGS. 8A and 8B). In the case of the combination with THCA, despite the detected slight increase in the CB2 agonistic activity, the difference was modest relative to treatment with curcumin alone (IC50 of 4,739±0,320 μM) (FIGS. 8A and 8B).

A deeper comparison study of the treatments was performed to further analyze the possible differences between curcumin and curcumin combination treatment. This study showed that THCA did not block the curcumin activation of CB2R (FIG. 9A). However, in curcumin-THC combination, at low curcumin concentration, THC did block the curcumin CB2R agonistic effect (FIG. 9B). At 10 μM curcumin and above, a potent CB2R agonist effect is observed which indicated that curcumin CB2R agonistic activity prevailed.

### Example 2D: Curcumin is an Inhibitor NF-κB Activity in Fibroblasts Both Alone and in Combination with Cannabinoids (THC, CBD)

The inhibitory activity of Curcumin alone and in combination with THC or CBD on TNFα-induced NF-κB activation was analyzed using the NIH-3T3-KBF-Luc cell line. Anti-NF-κB activity was reflected by the inhibition of TNFα-Luc activity as described in experimental design section.

As in Example 2A, five different concentrations of curcumin alone (0.1, 1, 5, 10 and 15 μM) or in the presence of 10 μM THC or 10 μM CBD were evaluated on anti NF-κB activity. Table 6 summarizes obtained data.

TABLE 6
Curcumin alone and curcumin combinations on NF-KB activity. Data are represented as percentage of activity.
A reduction of the percentage indicates anti NF-κB activity. Data are from independent experiments where the positive
control TNFα showed an induction of activity ≥10-fold. Only treatments that the mean of three consecutive
independent experiments shows a positive result (activity ≤50%) are considered inhibitory of NF-κB activity.

| | Experiment 1 | Experiment 2 | Experiment 3 | Conclusions: Is NF-κB |
|---|---|---|---|---|
| | % of activity | % of activity | % of activity | inhibitor effect observed? |
| TNFα 30 ng/mL (control without | 100.0 | 100.0 | 100.0 | Negative |
| inhibitor) | | | | |
| Curcumin 0.1 μM | 122.8 | 104.2 | 117.6 | Negative |
| Curcumin 1 μM | 130.7 | 99.2 | 129.2 | Negative |
| Curcumin 5 μM | 51.2 | 31.2 | 61.4 | + |
| Curcumin 10 μM | 0.3 | 3.3 | 17.8 | + |
| Curcumin 15 μM | −10.3 | −4.6 | −1.4 | + |
| THC 10 μM | 122.2 | 84.9 | 88.8 | Negative |
| Curcumin 0.1 μM + THC 10 μM | 123.9 | 74.3 | 94.5 | Negative |
| Curcumin 1 μM + THC 10 μM | 112.2 | 72.6 | 78.8 | Negative |
| Curcumin 5 μM + THC 10 μM | 30.5 | 19.0 | 21.2 | + |
| Curcumin 10 μM + THC 10 μM | −5.5 | −1.3 | 9.3 | + |
| Curcumin 15 μM + THC 10 μM | −9.1 | −2.9 | 4.6 | + |
| CBD 10 μM | 135.7 | 86.4 | 106.1 | Negative |
| Curcumin 0.1 μM + CBD 10 μM | 123.5 | 99.1 | 135.5 | Negative |
| Curcumin 1 μM + CBD 10 μM | 128.1 | 88.9 | 119.4 | Negative |
| Curcumin 5 μM + CBD 10 μM | 39.7 | 19.8 | 65.5 | + |
| Curcumin 10 μM + CBD 10 μM | −5.7 | −5.3 | 3.5 | + |
| Curcumin 15 μM + CBD 10 μM | −11.9 | −7.0 | −0.2 | + |

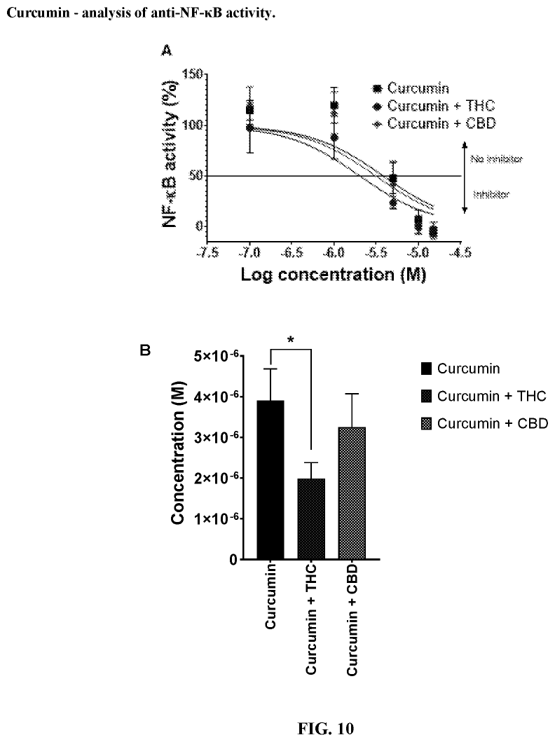

Curcumin alone showed a strong dose response effect against NF-κB demonstrating an anti-NF-κB IC50 value of 3,907±0,773 μM (FIG. 10A). The curcumin-CBD combination did not effect the anti-NF-κB activity relative to curcumin alone treatment (IC50 of 3,254±0,820 The inhibitory effect of curcumin was slightly improved when it was combined with THC (FIGS. 10A and 10B).

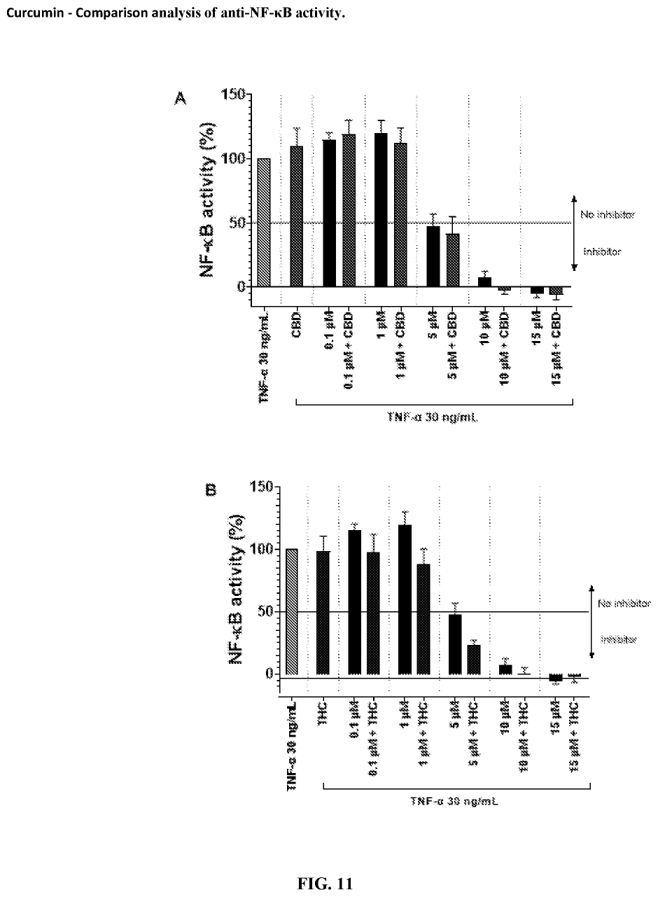

A deeper comparison study of the treatments was performed to further analyze the possible differences between curcumin and curcumin combinations treatment. THC and CBD did not modify the strong curcumin effect in any of the tested concentrations (FIGS. 11A and 11B). This result suggests that the curcumin component of the combinations of the invention will exert undiminished effects through NF-κ inhibition despite the presence of THC and/or CBD. Therapeutic uses of the invention may be deduced on this basis by those skilled in the art.

### Example 3: Animal Model of PK/PD and Bioavailability

Oral formulations of the invention were tested to determine key pharmacokinetic (PK) parameters and to ensure satisfactory exposure over time. PK assays are used to identify plasma concentration over time, area under the curve (AUC) exposure over 24 hrs, systemic clearance rate (CL) and systemic bioavailability (% F). The combination is also tested against the individual components. The 24 hr exposure identifies if the UDF should be administered QD (once a day) or BID (×2 a day) or more often, or less often.

Standard PK models are widely available and can be performed with a commercial service. A preferred method is to use at least 4 Male Sprague Dawley rats (210-230 g) who receive either an intravenous (i.v. 2, 5, and/or 10 mg/kg) or oral (5, 10 and/or 20 mg/kg) dose of each compound separately, or combined in formulation. Blood, urine, cerebrospinal fluid (CSF) or other appropriate biological fluid is removed at periodic intervals. The biological fluid is tested for active compound(s) in order to construct concentration vs. time profiles. These data are analyzed and pharmacokinetic parameters are calculated in order to assess in vivo pharmacokinetic activity.

The study uses a fixed dose of each component in the combination in a fixed vehicle formulation. In one embodiment PEG (polyethylene-glycol) is an excipient, or alternatively a long chain fatty acid oil carrier. Typically, components are prepared from a powder form, first in 5% ETOH, then with 40% PEG. The components are combined and topped up with distilled water to 100% volume. If components are not completely soluble, PEG may be increased to 60% and also add 10% PG.

By way of example, a PK study may be conducted at 10 mg/kg dose for an oral formulation. 2 mg/kg may be used for IV injection as a comparison. In either case, plasma samples are collected over a 24 hr time-period to determine bioavailability. Plasma samples are tested by HPLC or LC-MS/MS to obtain PK parameters e.g. 3 rats per each route of administration (total n=6) is typically sufficient.

Plasma and other tissue samples are tested for the administered cannabinoids and the curcumin administered to the animal. The samples are also tested for significant metabolites, some of which may have more potent effects than the parent administered compounds. The samples may also be used to determine baseline levels of serum biomarkers which are relevant to the development or treatment of the complex disease models set out further below. Many serum biomarkers are of great interest in the development or treatment of complex disorders. Biomarkers of interest to the compositions of the invention include IL-6, NF-kB, TNF-α, C-reactive protein, and any other biomarker known to be or potentially implicated in the development of a disease or disorder.

### Animal Models of Complex Disease or Condition

Compositions of the invention are tested in models corresponding to the disease and/or conditions proposed for use. These may be selected from among models of anxiety, pain, sleep induction, calmness induction, alertness induction, weight control, weight loss, obesity, diabetes and metabolic syndrome.

For any of the animal assays herein (including human testing), successful treatment may be identified according to the behavioural results identified in the assay, or by measuring biomarkers of disease progression/treatment, such as IL-6, NF-kB, TNF-α, C-reactive protein, and any other biomarker known to be or potentially implicated in the development of the disease or disorder being studied. Those skilled in the art are familiar with the wide variety of animal models available for further testing the products of the invention.

In summary, Examples 1-3 above provide evidence from bioinformatics, from in vitro assays and from in vivo mammalian experimentation, respectively, that the combinations of the invention have surprising and unexpected effects from which therapeutic utility, including synergistic therapeutic effects, are determined. Based on these discoveries, the inventors have further refined their analysis to provide preferred embodiments of the formulations more generally disclosed above.

### Example 4: Unit Dosage Form (UDF) Oral Capsule Embodiments

### Example 4-1—TimeWarp A3 Capsule (HardGel; Low-Dose THCA; Low Dose THC; Low Dose Curcumin; 370 mg Volume)

| Ingredient | Component | Final |
|---|---|---|
| Dried Cannabis | | 83.3 mg |
| THCA | 9 mg | |
| THC | 1 mg | |
| Curcumin | | 200 mg |
| Filler/stabilizer/anti-oxidant | | 86.7 mg |
| Net weight of capsule contents | | 370 mg |
| Capsule (HardGel Size 2) | | 62 mg |
| Gross weight of capsule | | 432 mg |
| (estimated) | | |

### Example 4-2—TimeWarp A3 Capsule (HardGel; Low-Dose THC; Low Dose Curcumin; 370 mg Volume)

| Ingredient | Component | Final |
|---|---|---|
| Dried Cannabis | | 83.3 mg |
| THC | 10 mg | |
| Curcumin | | 200 mg |
| Filler/stabilizer/anti-oxidant | | 86.7 mg |
| Net weight of capsule contents | | 370 mg |
| Capsule (HardGel Size 2) | | 62 mg |
| Gross weight of capsule | | 432 mg |
| (estimated) | | |

### Example 4-3—Island Mist/Timewarp A3 Capsule (HardGel; Low-Dose THC; Low Dose CBD; Low Dose Curcumin; 500 mg Capsule Volume)

| Ingredient | Component | Final |
|---|---|---|
| Dried Cannabis | | 166.6 mg |
| CBD | 10 mg | |
| THC | 10 mg | |
| Curcumin | | 200 mg |
| Filler/stabilizer/anti-oxidant | | 133.4 mg |
| Net weight of capsule contents | | 500 mg |
| Capsule (HardGel Size 1) | | 77 mg |
| Gross weight of capsule | | 577 mg |
| (estimated) | | |

### Example 4-4—Island Mist/Timewarp A3 Capsule (HardGel; Low-Dose THC; Low Dose CBD; High Dose Curcumin; 680 mg Volume)

| Ingredient | | Final |
|---|---|---|
| Dried Cannabis | | 166.6 mg |
| CBD | 10 mg | |
| THC | 10 mg | |
| Curcumin | | 400 mg |
| Filler/stabilizer/anti-oxidant | | 113.4 mg |
| Net weight of capsule contents | | 680 mg |
| Capsule (HardGel Size 0) | | 97 mg |
| Gross weight of capsule | | 777 mg |
| (estimated) | | |

### Example 4-5—1:1 CBD/THC; High Dose Curcumin; Hard-Gel Capsule; 500 mg Volume

| Ingredient | | Final |
|---|---|---|
| Dried Cannabis | | 166.6 mg |
| CBD | 10 mg | |
| THC | 10 mg | |
| Curcumin | | 323.4 mg |
| Filler/stabilizer/anti-oxidant | | 10 mg |
| Net weight of capsule contents | | 500 mg |
| Capsule (HardGel Size 1) | | 48 mg |
| Gross weight of capsule | | 548 mg |
| (estimated) | | |

### Example 4-6—High CBD; 4:1 CBD/THC; High Dose Curcumin; Hard-Gel Capsule; 500 mg Volume

| Ingredient | | Final |
|---|---|---|
| Dried Cannabis | | 166.6 mg |
| CBD | 25 mg | |
| THC | 1 mg | |
| Curcumin | | 323.4 mg |
| Filler/stabilizer/anti-oxidant | | 10 mg |
| Net weight of capsule contents | | 500 mg |
| Capsule (HardGel Size 1) | | 48 mg |
| Gross weight of capsule | | 548 mg |
| (estimated) | | |

### Example 4-7—Purple x Chemo Capsule (4:1 THC/CBG; High Dose Curcumin Hard-Gel Capsule; 500 mg Volume)

| Ingredient | | Final |
|---|---|---|
| Dried Cannabis | | 100 mg |
| THC | 10 mg | |
| CBG | 3 mg | |
| Curcumin | | 323.4 mg |
| Filler/stabilizer/anti-oxidant | | 76.6 mg |
| Net weight of capsule contents | | 500 mg |
| Capsule (HardGel Size 1) | | 48 mg |
| Gross weight of capsule | | 548 mg |
| (estimated) | | |

### Example 4-8—1:1 THC/CBD; Low Dose Curcumin Soft-Gel Capsule; 350 mg Volume

| Ingredient | | Final |
|---|---|---|
| Cannabis extract | | 33.3 mg |
| (60% cannabinoid in carrier) | | |
| THC | 10 mg | |
| CBD | 10 mg | |
| Curcumin | | 200 mg |
| (liquid format) | | |
| Carrier oil (Omega-3 to Omega-6 | | 66.7 mg |
| ratio greater than 1) with anti-oxidant | | |
| Net weight of capsule contents | | 300 mg |
| Capsule (Soft-gel) | | 50 mg |
| Gross weight of capsule | | 350 mg |

### Example 4-9—1:10 THC/CBD; Low Dose Curcumin Soft-Gel Capsule; 300 mg Volume

| Ingredient | | Final |
|---|---|---|
| Cannabis extract | | 18.3 mg |
| (60% cannabinoid in carrier) | | |
| THC | 1 mg | |
| CBD | 10 mg | |
| Curcumin | | 200 mg |
| (liquid format) | | |
| Carrier oil (Omega-3 to Omega-6 | | 31.7 mg |
| ratio greater than 1) with anti-oxidant | | |
| Net weight of capsule contents as | | 250 mg |
| formulated in nano-emulsion | | |
| Capsule (Soft-gel) | | 50 mg |
| Gross weight of capsule | | 300 mg |

### Example 4-10—10:2:1 THC/CBG/CBC; Low Dose Curcumin Soft-Gel Capsule; 273 mg Volume

| Ingredient | | Final |
|---|---|---|
| Cannabis extract | | 21.7 mg |
| (60% w/w) | | |
| THC | 10 mg | |
| CBG | 2 mg | |
| CBC | 1 mg | |
| Other cannabis extract ingredients | 8.7 mg | |
| included in liquid extract: residual | | |
| solvent, lipids, waxes, sugars, and | | |
| other phytochemicals and | | |
| antioxidants | | |
| Curcumin (liquid format) | | 80 mg |
| Carrier oil having Omega-3 to | | 171.3 mg |
| Omega-6 ratio at least 1.0 | | |
| Net weight of capsule contents | | 273 mg |
| Capsule (SoftGel Size 3) | | 50 mg |
| Gross weight of capsule | | 323 mg |
| (estimated) | | |

### Example 4-11—2:1:1 THC/CBG/CBC; Low Dose Curcumin Soft-Gel Capsule; 273 mg Volume

| Ingredient | | Final |
|---|---|---|
| Cannabis extract | | 20 mg |
| (60% w/w) | | |
| THC | 6 mg | |
| CBG | 3 mg | |
| CBC | 3 mg | |
| Other cannabis extract ingredients | 8 mg | |
| included in liquid extract: residual | | |
| solvent, lipids, waxes, sugars, and | | |
| other phytochemicals and | | |
| antioxidants | | |
| Curcumin (liquid format) | | 80 mg |
| Carrier oil having Omega-3 to | | 173 mg |
| Omega-6 ratio at least 1.0 | | |
| Net weight of capsule contents | | 273 mg |
| Capsule (SoftGel Size 3) | | 50 mg |
| Gross weight of capsule | | 323 mg |
| (estimated) | | |

### Example 4-12—2:1:1 CBD/CBG/CBC; Low Dose Curcumin Soft-Gel Capsule; 273 mg Volume

| Ingredient | | Final |
|---|---|---|
| Cannabis extract | | 20 mg |
| (60% w/w) | | |
| CBD | 6 mg | |
| CBG | 3 mg | |
| CBC | 3 mg | |
| Other cannabis extract ingredients | 8 mg | |
| included in liquid extract: residual | | |
| solvent, lipids, waxes, sugars, and | | |
| other phytochemicals and | | |
| antioxidants | | |
| Curcumin (liquid format) | | 80 mg |
| Carrier oil having Omega-3 to | | 173 mg |
| Omega-6 ratio at least 1.0 | | |
| Net weight of capsule contents | | 273 mg |
| Capsule (SoftGel Size 3) | | 50 mg |
| Gross weight of capsule | | 323 mg |
| (estimated) | | |

While the invention covers all oral formulations described herein, specific attention is drawn to the combination of curcumin with those cannabinoids in the amount set out in Table 1A, 1B and 1C. Further preferred combinations include but are not limited to: a fixed dose curcumin+CBD:CBG combination for the treatment of neuropathic pain; combinations of curcumin+THCA:CBD, curcumin+THC:CBD and curcumin+THC:CBDA for treatment of chronic pain.

Oral softgel capsule formulations comprising fixed dose active pharmaceutical ingredients listed in the examples are especially preferred. Treatment with such combinations provides analgesic and anti-inflammatory effects without the recognized adverse side effects associated with NSAID use.

Further combinations of the invention are provided in Table 7

TABLE 7
Preferred Defined Dose combination products of the invention

| | | | | | | | | | | Final | |
|---|---|---|---|---|---|---|---|---|---|---|---|
| | | | | | | | | | | capsule | |
| Prod | | Capsule | Defined Dose (mg) | | | | | | | mass* | |
| # | Capsule | Fill | Curcumin | THC | THCA | THCV | CBD | CBG | CBC | (mg) | Indication |
| 4-1 | HardGel | Dry | 200 | 1 | 9 | | | | | 432 | Pain, Nausea, |
| | | | | | | | | | | | Obesity, |
| | | | | | | | | | | | Metabolic |
| | | | | | | | | | | | Syndrome, |
| | | | | | | | | | | | Inflammation |
| 4-2 | HardGel | Dry | 200 | 10 | | | | | | 432 | Pain, Appetite |
| | | | | | | | | | | | enhancement, |
| | | | | | | | | | | | Inflammation; |
| 4-3 | HardGel | Dry | 200 | 10 | | | 10 | | | 577 | Pain; Anxiety; |
| | | | | | | | | | | | Sleep, |
| | | | | | | | | | | | Inflammation; |
| 4-4 | HardGel | Dry | 400 | 10 | | | 10 | | | 777 | Pain; Anxiety; |
| | | | | | | | | | | | Sleep, |
| | | | | | | | | | | | Inflammation; |
| 4-5 | HardGel | Dry | 323.4 | 10 | | | 10 | | | 548 | Pain; Anxiety; |
| | | | | | | | | | | | Sleep, |
| | | | | | | | | | | | Inflammation; |
| 4-6 | HardGel | Dry | 323.4 | 1 | | | 25 | | | 548 | Anti-epileptic, |
| | | | | | | | | | | | Chronic Pain, |
| | | | | | | | | | | | Inflammation, |
| | | | | | | | | | | | Schizophrenia, |
| | | | | | | | | | | | Diabetes |
| 4-7 | HardGel | Dry | 323.4 | 10 | | | | 3 | | 548 | Pain; Nausea, |
| | | | | | | | | | | | Inflammation, |
| | | | | | | | | | | | Appetite |
| | | | | | | | | | | | stimulation |
| 4-8 | SoftGel | Oil with | 200 | 10 | | | 10 | | | 350 | Pain; Anxiety; |
| | | Omega-3 to | | | | | | | | | Sleep, |
| | | Omega-6 | | | | | | | | | Inflammation |
| | | ratio of at | | | | | | | | | |
| | | least 1.0 | | | | | | | | | |
| 4-9 | SoftGel | Oil with | 200 | 1 | | | 10 | | | 300 | Anti-epileptic, |
| | | Omega-3 to | | | | | | | | | Chronic Pain, |
| | | Omega-6 | | | | | | | | | Inflammation, |
| | | ratio of at | | | | | | | | | Schizophrenia; |
| | | least 1.0 | | | | | | | | | Diabetes |
| 4-10 | SoftGel | Surfactant | 200 | 1 | | | 10 | | | 300 | Anti-epileptic, |
| | | (i.e. | | | | | | | | | Chronic Pain, |
| | | Labrasol ™) | | | | | | | | | Inflammation, |
| | | plus Oil | | | | | | | | | Schizophrenia; |
| | | with | | | | | | | | | Diabetes |
| | | Omega-3 to | | | | | | | | | |
| | | Omega-6 | | | | | | | | | |
| | | ratio of at | | | | | | | | | |
| | | least 1.0 | | | | | | | | | |
| 4-11 | SoftGel | Oil with | 200 | | | 10 | 10 | | | 548 | Pain; Anxiety; |
| | | Omega-3 to | | | | | | | | | Sleep, |
| | | Omega-6 | | | | | | | | | Inflammation; |
| | | ratio of at | | | | | | | | | Diabetes; Appetite |
| | | least 1.0 | | | | | | | | | supression |
| 4-12 | HardGel | Dry | 323.4 | 5 | | | 10 | | | 548 | Anti-epileptic, |
| | | | | | | | | | | | Chronic Pain, |
| | | | | | | | | | | | Inflammation, |
| | | | | | | | | | | | Schizophrenia, |
| | | | | | | | | | | | Diabetes |
| 4-13 | SoftGel | Surfactant | 200 | 5 | | | 10 | | | 250 | Anti-epileptic, |
| | | (i.e. | | | | | | | | | Chronic Pain, |
| | | Labrasol ™) | | | | | | | | | Inflammation, |
| | | plus Oil | | | | | | | | | Schizophrenia, |
| | | with | | | | | | | | | Diabetes |
| | | Omega-3 to | | | | | | | | | |
| | | Omega-6 | | | | | | | | | |
| | | ratio of at | | | | | | | | | |
| | | least 1.0 | | | | | | | | | |
| 3-18 | SoftGel | Oil with | 180 | 10 | | | | 2 | 1 | 250 | Pain, |
| | | Omega-3 to | | | | | | | | | Inflammation, |
| | | Omega-6 | | | | | | | | | Gastrointestinal |
| | | ratio of at | | | | | | | | | disorders, |
| | | least 1.0 | | | | | | | | | Neurodegenerative |
| | | | | | | | | | | | disorders |
| 3-19 | SoftGel | Oil with | 180 | 6 | | | | 3 | 3 | 250 | Pain, |
| | | Omega-3 to | | | | | | | | | Inflammation, |
| | | Omega-6 | | | | | | | | | Gastrointestinal |
| | | ratio of at | | | | | | | | | disorders, |
| | | least 1.0 | | | | | | | | | Neurodegenerative |
| | | | | | | | | | | | disorders |
| 3-20 | SoftGel | Oil with | 180 | | | | 6 | 3 | 3 | 250 | Pain, |
| | | Omega-3 to | | | | | | | | | Inflammation, |
| | | Omega-6 | | | | | | | | | Gastrointestinal |
| | | ratio of at | | | | | | | | | disorders, |
| | | least 1.0 | | | | | | | | | Neurodegenerative |
| | | | | | | | | | | | disorders |

*including capsule shell and all carrier, filler, stabilizer, and anti-oxidant, etc.

Any of the capsules provided herein may include an extended release (enteric) coating. An example of a suitable enteric coating is provided in Table 8.

TABLE 8
Optional extended release coating for use with capsules of the invention.

| Component | Function | % w/w | mg/capsule |
|---|---|---|---|
| Ethylcellulose dispersion | Water-insoluble film- | 71.4 | 67 |
| (Aquacoat ECD 30) | forming polymer | | |
| Triethyl citrate | Plasticizer | 14.3 | 13 |
| Polyvinyl alcohol/ | Water-soluble pore | 14.3 | 13 |
| polyethylene glycol | former | | |
| co-polymer (Kollicoat IR) | | | |
| Total | | 100.0% | 93 mg |

While preferred embodiments of the present invention have been shown and described herein, those skilled in the art recognize that such embodiments are provided by way of example only. Numerous variations, changes, and substitutions will occur to those skilled in the art without departing from the invention. It should be understood that various alternatives to the embodiments of the invention described herein may be employed in practicing the invention. It is intended that the following claims define the scope of the invention and that methods and formulations within the scope of these claims and their equivalents be covered thereby.

What is claimed is:

1. An oral formulation comprising:
a. one or more cannabinoids selected from among the group consisting of:
0.1-750 mg tetrahydrocannabinolic acid (THCA),
0.1-100 mg tetrahydrocannabinol (THC),
0.1-750 mg cannabidiolic acid (CBDA),
0.1-750 mg cannabidiol (CBD),
0.1-750 mg cannabichromene (CBC),
0.1-750 mg cannabigerol (CBG); and
b. at least one of: curcumin; and any other curcuminoid.

2. The oral formulation of claim 1 in a unit dosage form selected from the group consisting of a pill, tablet, capsule, film, wafer, lollipop, lozenge, oil, tincture, and syrup.

3. The oral formulation of claim 2, wherein the formulation is an orally disintegrating film, or wafer.

4. The oral formulation of claim 1 or 2, wherein the formulation is a pill or tablet and further comprises an enteric coating for containing the one or more cannabinoids and the lipid carrier.

5. The oral formulation of any one of claim 1, 2 or 4, wherein the formulation is a pill, tablet, or capsule, and further comprises an outer shell that is substantially opaque to one or both of ultraviolet and visible light.

6. The oral formulation of any one of claim 1, 2, 4 or 5 further comprising a carrier oil.

7. The oral formulation of any one of claims 1 to 6 further comprising a surfactant.

8. The oral formulation of any one of claims 1 to 7, wherein one or more of the cannabinoids is present in the form of an organic solvent-based extract of Cannabis.

9. The oral formulation of any one of claims 1, 2 or 4 to 6, wherein one or more of the cannabinoids is present in the form of dried Cannabis flower.

10. The oral formulation of any one of claims 1 to 9, wherein one or more of the cannabinoids is the product of a biosynthetic process in yeast, a microbe, a non-Cannabis cell-based system or a cell-free system.

11. The oral formulation of any one of claims 1 to 10, wherein the formulation comprises a defined dose or combination dose of cannabinoid(s) selected from the list consisting of (each cannabinoid milligram amount about or equal to):
THC (10 mg), CBD (10 mg), and curcumin (200 mg),
THC (10 mg), CBG (3 mg), and curcumin (200 mg),
THC (1 mg), CBD (25 mg), and curcumin (400 mg),
THC (10 mg), CBD (10 mg), and curcumin (323.4 mg),
THC (5 mg), CBD (20 mg), and curcumin (323.4 mg),
THC (10 mg), CBG (3 mg), and curcumin (323.4 mg),
THC (1 mg), CBD (10 mg), and curcumin (200 mg),
THC (1 mg), THCA (9 mg), and curcumin (200 mg),
THC (10 mg) and curcumin (200 mg),
THC (10 mg), CBD (10 mg), and curcumin (200 mg),
THC (10 mg), CBD (10 mg), and curcumin (400 mg),
THC (10 mg), CBD (10 mg), and curcumin (323.4 mg),
THC (1 mg), CBD (25 mg), and curcumin (323.4 mg),
THC (10 mg), CBG (3 mg), and curcumin (323.4 mg),
THC (10 mg), THCV (10 mg), and curcumin (200 mg),
THCV (10 mg), CBD (10 mg), and curcumin (200 mg),
THC (5 mg), CBD (10 mg), and curcumin (323.4 mg),
THC (5 mg), CBD (10 mg), and curcumin (200 mg),
THC (10 mg), CBG (3 mg), CBC (3 mg) and curcumin (200 mg),
THC (10 mg), CBG (3 mg), CBC (3 mg) and curcumin (400 mg),
THC (10 mg), CBG (2 mg), CBC (1 mg) and curcumin (180 mg),
.THC (6 mg), CBG (3 mg), CBC (3 mg) and curcumin (180 mg), and
CBD (6 mg), CBG (3 mg), CBC (3 mg) and curcumin (180 mg).

12. The oral formulation of any one of claims 1 to 11, further comprising at least one further cannabinoid selected from the group consisting of CBN (cannabinol), CBG, CBGA, CBC, and tetrahydrocannabivarin (THCV).

13. The oral formulation of any one of claims 1 to 12, comprising CBD in an amount between 10-50 mg.

14. The oral formulation of claim 13, comprising 25 mg CBD.

15. The oral formulation of any one of claims 1 to 14, comprising 500 mg CBD.

16. The oral formulation of any one of claims 1 to 15, wherein curcumin is present in an amount between about 50 mg and about 400 mg.

17. The oral formulation of claim 16, wherein the dose of curcumin is present in an amount of about 100 mg, about 200 mg, or about 400 mg.

18. The oral formulation of claim 16, wherein the curcumin is present in the form of an organic solvent-based extract.

19. The oral formulation of any one of claims 1 to 18, wherein the cannabinoid is physically separated from the curcumin.

20. The oral formulation of any one of claims 1 to 18, wherein the cannabinoid is evenly dispersed within at least a portion of the oral formulation.

21. The oral formulation of any one of claims 1 to 20, wherein a signifier which signifies the cannabinoid dosage is associated directly with the oral formulation by embossing, or by colour, pattern or shape feature.

22. The oral formulation of claim 21, wherein the signifier is adapted to be directly interpreted by a consumer and/or is a machine-readable code.

23. The oral formulation of any one of claims 1 to 22, wherein the oral formulation is contained in an individual blister pack sealed in an inert gas atmosphere comprising little or no oxygen.

24. The oral formulation of any one of claims 1-2, 4-8 or 10-23, further comprising a softgel containing the one or more cannabinoids and/or the curcumin in a liquid fill.

25. The oral formulation of claim 24, wherein the liquid fill comprises a mixture of carrier oil and liquid curcumin.

26. The oral formulation of claim 25 wherein the carrier oil comprises an oil having a lipid ratio by weight of Omega-3 to Omega-6 of 1 or higher.

27. The oral formulation of claim 26 wherein the liquid fill further comprises a surfactant.

28. The oral formulation of any one of claims 1 to 27, comprising curcumin.

29. The oral formulation of any one of claims 1 to 28, wherein the one or more cannabinoids, and/or the curcuminoid, is present in a defined dose.

30. A method of administering a cannabinoid to an individual, the method comprising administering curcumin to the individual in combination with the cannabinoid, in an oral formulation of any one of claims 1 to 29.

31. The method of claim 30, wherein the individual is suffering from one or more diseases, conditions or disorders selected from the group consisting of pain, inflammation, anxiety, depression, sleep disorders, insomnia, lack of energy, lack of alertness, weight gain, obesity, diabetes, Metabolic Syndrome, acute and anticipatory nausea, suppressed appetite, epilepsy, spasticity, schizophrenia, bi-polar disorder, cancer and neoplasia, chronic pain, osteoarthritic pain, bacterial and/or fungal infection and fibromyalgia.

32. The method of claim 31, wherein the administering results in amelioration and/or treatment of one or more symptoms associated with the one or more diseases, conditions, or disorders.

33. A method of treating an individual suffering from one or more diseases, conditions or disorders selected from the group consisting of pain, inflammation, anxiety, depression, sleep disorders, insomnia, lack of energy, lack of alertness, weight gain, obesity, diabetes, Metabolic Syndrome, acute and anticipatory nausea, suppressed appetite, epilepsy, spasticity, schizophrenia, bi-polar disorder, cancer and neoplasia, chronic pain, osteoarthritic pain, bacterial and/or fungal infection and fibromyalgia, the method comprising administration to the individual of a therapeutically effective amount of an oral formulation of any one of claims 1 to 29.

34. A method of manufacturing an oral formulation of any one of claims 1 to 29, comprising:
a. providing an organic extract of cannabinoids from cultivated Cannabis,
b. measuring the concentration of one or more cannabinoids selected from the group consisting of THCA, THC, CBDA and CBD in the organic extract,
c. adjusting the concentration of one or more cannabinoids in the extract to prepare an adjusted extract within the defined dose tolerance limits of a manufacturing specification for the oral formulation; and
d. manufacturing the oral formulation with the adjusted extract.

35. A method of manufacturing an oral formulation of any one of claims 1 to 29, comprising
a. grinding a sample of Cannabis such that it passes through a mesh of average opening size of about 0.595 mm, about 0.250 mm, or about 0.125 mm to produce a ground sample,
b. measuring the concentration of one or more cannabinoids selected from the group consisting of THCA, THC, CBDA, CBC, and CBD in the ground sample,
c. adjusting the concentration of one or more cannabinoids in the ground sample to prepare an adjusted ground sample within the defined dose tolerance limits of a manufacturing specification for the oral formulation;
d. manufacturing the oral formulation with the adjusted ground sample.

36. Use of the oral formulation of any one of claims 1 to 29 for treating a disease or disorder in a subject in need thereof.

37. The use of claim 36, wherein the disease or disorder is selected from the group consisting of pain, inflammation, anxiety, depression, sleep disorders, insomnia, lack of energy, lack of alertness, weight gain, obesity, diabetes, Metabolic Syndrome, acute and anticipatory nausea, suppressed appetite, epilepsy, spasticity, schizophrenia, bi-polar disorder, cancer and neoplasia, chronic pain, osteoarthritic pain, bacterial and/or fungal infection and fibromyalgia.

38. A method of preparing an oral formulation, the method comprising:
a. preparing or obtaining a ground or milled Cannabis plant material preparation;
b. passing the Cannabis plant material preparation through a mesh or sieve to obtain a retained fraction of the material which is retained in the mesh or sieve and a pass-through fraction of the material which has passed through the mesh or sieve;
c. determining the content of the one or more cannabinoids of interest in the retained fraction and/or the pass-through fraction;
d. based on the content determined in step (c), determining a target amount of retained fraction material, pass-through material, or a combination thereof, to be incorporated into the formulation such that the formulation includes the desired defined dose of the one or more desired cannabinoids; and
e. combining the target amount of retained fraction material, pass-through material, or combination thereof with curcumin, thereby forming the formulation.

39. The oral formulation of any one of claims 1 to 29, wherein the oral formulation comprises a ground or milled Cannabis plant material preparation which in turn comprises the one or more cannabinoids, wherein the preparation is prepared by a process comprising the following steps:
a. preparing or obtaining a ground or milled Cannabis plant material preparation;
b. passing the Cannabis plant material preparation through a mesh or sieve to obtain a retained fraction of the material which is retained in the mesh or sieve and a pass-through fraction of the material which has passed through the mesh or sieve;
c. determining the content of the one or more cannabinoids of interest in the retained fraction and/or the pass-through fraction; and
d. based on the content determined in step (c), determining a target amount of retained fraction material, pass-through material, or a combination thereof, to be incorporated into the formulation such that the formulation includes the desired defined dose of the one or more desired cannabinoids.
